(19) United States
(12) Patent Application Publication
(10) Pub. No.: US 2002/0106671 A1
(43) Pub. Date: Aug. 8, 2002

(54) Identification of a capacitative calcium channel in antigen presenting cells and uses thereof

(76) Inventors: Veronique Schindler, Paris (FR); Carole Bloes, Orsay (FR); Michel Fink, Fresnes (FR); Janet Allen, Meudon (FR); Guido Grentzmann, Verrieres Le Buisson (FR)

Correspondence Address: NIXON & VANDERHYE P.C., 8th Floor, 1100 North Glebe Road, Arlington, VA, 22201, US

(21) Appl. No.: 09/946,175

(22) Filed: Sep. 5, 2001

(30) Foreign Application Priority Data
Sep. 5, 2000 (EP) 00 402436.0

(51) Int. Cl. C12Q 1/68

(52) U.S. Cl. 435/6

(57) ABSTRACT

The present invention relates to the identification of a capacitative calcium channel homologue for the immune system activation, and its use to report and to modulate modulate the activity of immune cells in vitro, ex vivo or in vivo. This invention more specifically discloses that a Mlsn1 gene product, represents a capacitative calcium channel in immune cells such as macrophages, monocytes, T-cells, B-cells and mast-cells. This invention is a proven identification of a gene expressing a capacitative calcium channel in immune cells, and can be used in various compositions and methods for monitoring or modulating an immune response in a subject. The present invention can be used to develop biomarkers for immune system activation or inflammatory responses or to screen for specific immune system activity altering drugs.

### FIELD OF INVENTION

The present invention relates to the caracterisation of a capacitative calcium channel homologue for the immune system activation, and its use to report or to modulate the activity of immune cells in vitro, ex vivo or in vivo. This invention more specifically discloses that a mlsn1 gene product represents a capacitative calcium channel in immune cells such as macrophages, monocytes, T-cells, B-cells and mast-cells. This invention is a proven identification of a gene expressing a capacitative calcium channel in immune cells, and can be used in various compositions and methods for monitoring or modulating an immune response in a subject. The present invention can be used to develop biomarkers for immune system activation or inflammatory responses or to screen for specific immune system activity altering drugs.

### BACKGROUND OF THE INVENTION

Regulated entry of calcium across the plasma membrane is an essential signaling mechanism, implicated in phenomena like exocytosis, contraction, gene expression, and cell differentiation (1). Store operated channels (SOCs), defined as channels that open in response to depletion of intra-cellular calcium stores, represent one of the most ubiquitous mechanisms for triggering calcium influx in non-excitable cells.

A large number of cell surface receptors are coupled through G-proteins or tyrosine kinases to the activation of phospholipase C (PLC) and consequently the generation of inositol 1,4,5-triphosphate (IP3) (2). Increased IP3, acting on its receptor (3) results in an increase of intra-cellular calcium ([Ca²⁺]i) through depletion of intra-cellular calcium stores. How depletion of intra-cellular calcium stores triggers Ca²⁺ entry through the cell membrane is still controversial. The simplest mechanism would involve a direct action of IP3, or of the IP3 receptor, on Ca²⁺ channels in the plasma membrane. On the other hand depletion of stores might send an activating signal of some kind to channels in the plasma membrane (4). Plasma membrane Ca²⁺ channel activation results in a small inward current in parallel with a sustained raise in [Ca²⁺]i. Such a current has been shown to be induced by intra-cellular dialysis with IP3 in rat mastcells (5) or by Thapsigargin-activation of Jurkat T-cells (6). Thapsigargin (TG), a plant derived sesquiterpene lactone, depletes IP3-sensitive stores through its ability to inhibit Ca²⁺-ATPases (7). Several groups have shown that TG depletion of intracellullar calcium stores in T-cells activates a Ca²⁺ influx that was similar to and not additive with that evoked by T-Cell Receptor (TCR) stimulation (8-10).

SOCs have a high diversity. The essential defining feature of these channels is that they are activated by a variety of stimuli that deplete intra-cellular stores such as agonists to phosphoinositide-linked receptors, intra-cellular IP3, Ca²⁺ ionophores, inhibitors of SERCA-type Ca²⁺ ATPases, and intra-cellular dialysis with buffered solutions containing low concentrations of Ca²⁺ ([Ca²⁺]<50 nM) (1). IP3 activation of SOCs implies that the channel is not directly coupled to the receptor and artificial store depletion activation indicates that SOC activation responds to store Ca²⁺ depletion after IP3-receptor binding than to IP3 itself. SOCs are generally described as lacking voltage dependent gating.

Calcium entry through Icrac has been shown to accelerate the rate of exocytosis in mast cells. It inhibits adenylyl cyclase type VI, but activates adenylyl cyclase type I. Icrac activity might be regulated either directly or indirectly through the concentration of intra-cellular Ca²⁺ (13). An increase in the intra-cellular calcium ion concentration controls a diverse range of cell functions, including adhesion, mobility, gene expression and proliferation can occur as single transients, repetitive oscillations or a sustained plateau (14). Two recent reports have suggested that the amplitude, duration and frequency of a Ca²⁺ signal in cells of the immune system control differential activation of proinflammatory transcriptional regulators NF-κB, JNK and NF-AT (15; 16). Thus Calcium signalling could play a crucial role in the activation of cells of the immune system. The identification of an immune system specific Calcium channel would therefore represent an important step forward in determining the pathology of Icrac in disease states. Mlsn1 and its gene products could therefore be used as a basis of screens to identify new therapeutic compounds such as immune system suppressors, including anti-allergic or anti-inflammatory compounds, or immune system activators for the treatment of immuno degenerative disorders. Mlsn1 could as well represent a powerfull biomarker for onset and progression of autoimmune or inflammatory deseases (e.g. COPD, Arthritis, organ rejection).

### SUMMARY

The present invention discloses the caracterisation of a capacitative Ca²⁺ channel in immune cells, more specifically in macrophages. MRNA expression is differentially increased in monocytes to macrophages differentiation as well as in macrophages activation.

More particularly, the inventors have identified the presence of a novel calcium pore sequence within the products of the mlsn1 gene, and this calcium pore is part of the major capacitative calcium channel in immune system cells, more specifically in macrophages.

The present invention shows that a product of the mlsn1 gene is expressed in the U937 cells (monocyte like) specifically after 48 h differentiation to a more macrophage like cell type and that mlsn1 regulates capacitative calcium entry in these cells. Expression is further increased by macrophages activation. PCR subtraction experiments show that mlsn1 gene expression is several times upregulated, which is in concordance with the previously established differential increases of the Calcium signalling phenotype during this differentiation step (18).

This invention also shows that particular mlsn1-specific anti-sense nucleic acids down regulate calcium entry in U937 cells differentiated to a macrophage phenotype. This is of particular importance since it is the first time that a sequence specific tool has succeeded to inhibit the immune cell Calcium signalling, thereby associating a given gene to the Ca²⁺ pore gene expression.

This invention represents the caracterisation of a SOC channel in immuno-modulatory cells, more specifically in macrophages. This invention provides a mean 1) to use it as a modulator of immune activation 2) to use it as a biomarker for immune activation and inflammation 3) to identify new targets for the screening of immuno-modulators.

The described invention further shows, the nucleic acid sequence of the Mlsn1 transcriptional promoter elements 5′ to the putative Mlsn1 start codon.

An object of the present invention resides in the use of a mlsn1 gene, gene product(s) and transcription promoters for the screening of compounds that modulate the activity of an Icrac expressing immune cell, more specifically of an antigen presenting cell, most specifically of a compound that modulates the activity of macrophages.

Further objects of the present invention include Mlsn1 peptides, nucleic acids, vectors, and recombinant cells, which can be used for therapeutic (as target) or diagnostic approaches (as biomarker).

### BRIEF DESCRIPTION OF THE FIGURES

FIG. 1: Amino acid sequence of Mlsn1 with predicted secondary structure.

Where;<<N-TERMINUS>> represents the N-terminal domain

_TMD1 to 6 represent the transmembrane domains

—EXL1 to 3—represents the extra-cellular loop

—INL1 and 2—represent the intra-cellular loops

<<C-TERMINUS>> represents the C-terminal domain

FIG. 2: Represents the analysis on agarose of PCR products obtained with Mlsn1-specific primer pairs from differentiated or undifferentiated U937 macrophage cells.

FIG. 3: Illustrates the Mlsn1 (TrpC8) expression analysis by cDNA subtraction.

1: dbcAMP differentiated U937 subtracted by undifferentiated U937.

2: unsubtracted dbcAMP differentiated U937 control.

3: undifferentiated U937 subtracted by dbcAMP differentiated U937.

4: unsubtracted undifferentiated U937 control.

5: 1 kb ladder

PCR have been carried out on subtraction DNA fragments using “Not 1a” and “Not 2b” Mlsn1 specific primers.

FIG. 4: Down regulation of calcium entry during dbcAMP differentiation of U937 cells using Mlsn1 anti-sense-Calcium flux analysis using Indo-1 and flow cytometry

a: Undifferentiated U937

b: dbcAMP differentiated U937

c: dbcAMP differentiated U937 treated with 10 μM scrambled oligonucleotide

d: dbcAMP differentiated U937 treated with 1 μM Mlsn1 anti-sense

e: dbcAMP differentiated U937 treated with 10 μM Mlsn1 anti-sense

### DETAILED DESCRIPTION OF THE INVENTION

An object of the present invention resides in the caracterisation of functional components of Mlsn1. The polypeptide structure of Mlsn1 has been determined. Nucleic acid sequences and peptides which encode the N- and C-terminal regions have been identified. Also, nucleic acid sequences and peptides which encode transmembrane domains and intra- and extra-cellular loop regions have been identified. Crucially, nucleic acid and peptide sequences which encode the Mlsn1 calcium pore region have been identified. The above nucleic acid and peptide sequences offer vital target molecules for the development of therapeutic molecules to combat disorders associated with aberrant Icrac function.

Furthermore, the inventors have identified putative transcription promoter elements 5′ to the nucleic acid encoding Methionine-1 start codon of Mlsn1.

Another object of this invention resides, generally, in the use of a mlsn1 gene, gene product, fragments thereof, or transcriptional promoter elements for the screening of compound(s) that modulate the activity of an immune cell, more specifically for the screening of a compound that modulates the activity of monocytes, macrophages, T-cells, B-cells and mast-cells in vitro, ex vivo or in vivo, most specifically for the screening of compounds that modulate the activity of macrophages in vitro, ex vivo or in vivo. This invention also relates to particular Mlsn1 polypeptides and nucleic acids, methods of screening of immuno-modulators.

Other aspects of the present invention relate to:

Methods of screening for immuno-modulatory compounds, comprising contacting a test compound with a Mlsn1 gene or a Mlsn1 gene product or Mlsn1 transcription promoter element(s) and determining the ability of said test compound to interact with the Mlsn1 gene or a Mlsn1 gene product or Mlsn1 transcriptional promoter element(s).

Methods of screening for identification of new (known or unknown) immuno-modulatory targets after modulation of the Mlsn1 gene, Mlsn1 gene product activity or transcription promoter activity.

Further aspects of the present invention relate to:

The use of mlsn1 gene, gene products or nucleic acids to report and/or to modulate the activity of Mlsn1 in immune cells in vitro, ex vivo or in vivo.

Within the context of the present invention, the term mlsn1 gene designates a nucleotide sequence encoding the Mlsn1 protein, any nucleotide sequence encoding a fragment of the Mlsn1 protein, or any variants or homologs thereof. Variants or homologs designate more particularly any naturally-existing human genes that would exhibit sequence variation(s) resulting from polymorphism(s), mutation(s), or other alteration(s) as compared to the above sequence, without depriving the property of the encoded protein to function as an Icrac channel. Homologs or variants also include any recombinant DNA molecule, such as a cDNA molecule, encoding a Mlsn1 protein. Further variants also include any sequence that would hybridize with the above nucleic acid sequences under stringent conditions and encode a functionally active Icrac channel. Nucleotide sequences encoding fragments of the Mlsn1 protein which represent a biological relevant region of this protein such as the N- and C- terminal domains, the transmembrane domains, the intra and extra-cellular loops and the predicted calcium pore as well as the polypeptides representing these biologically relevant regions are also included.

As an illustrative embodiment, stringent hybridization conditions can be defined as follows:

The hybridization step is conducted at 65° C. in the presence of 6×SSC buffer, 5×Denhardt's solution, 0.5% SDS and 100 μg/ml of salmon sperm DNA.

The hybridization step is followed by four washing steps:

two washings during 5 minutes, preferably at 65° C. in a 2×SSC and 0.1% SDS buffer;

one washing during 30 minutes, preferably at 65° C. in a 2×SSC and 0.1% SDS buffer;

one washing during 10 minutes, preferably at 35° C. in a 0.1×SSC and 0.1% SDS buffer,

it being understood that the hybridization conditions defined above are suitable for nucleic acids of approximately twenty nucleotides in length and that these conditions may be also adapted for shorter or longer nucleic acids, according to techniques well known in the art, for example those described by Sambrook et al. (1989).

A Mlsn1 gene product designates, in accordance with the present invention, polypeptides (or proteins) encoded by a mlsn1 gene as described above. The Mlsn1 gene product may comprise several polypeptides, resulting from alternative splicings of a mlsn1 gene during transcription. More specific Mlsn1 gene products of this invention are encoded by a mlsn1 gene as described above and/or comprise the amino acid sequence of Mlsn1, such as that of SEQ ID NO1.

Also within the context of the present invention, the term Mlsn1 transcription promoter(s) designates nucleotide sequence(s) encoding Mlsn1 transcription promoter element(s), nucleotide sequence encoding a fragment thereof, or any variants or homologs thereof. Variants or homologs designate more particularly any human transcription promoter elements that would exhibit sequence variation(s) resulting from polymorphism(s), mutation(s), or other alteration(s) as compared to the above sequence, without depriving the property of the nucleic acid sequences to function as an Icrac transcription promoter. Further variants also include any sequence that would hybridize with the above nucleic acid sequences under stringent conditions and encode a functionally active transcription promoter.

A Mlsn1 transcription promoter element designates, in accordance with the present invention, encoded by a Mlsn1 nucleic acid sequence as described above. The transcription promoter elements is further characterized by being located 5′ the Mlsn1 start codon. More specific Mlsn1 transcription promoter elements of this invention are encoded by MlsnI nucleic acid sequences of SEQ ID NO41, 42 or 43.

Nucleotide and Amino Acid Sequence Encoding Mlsn1

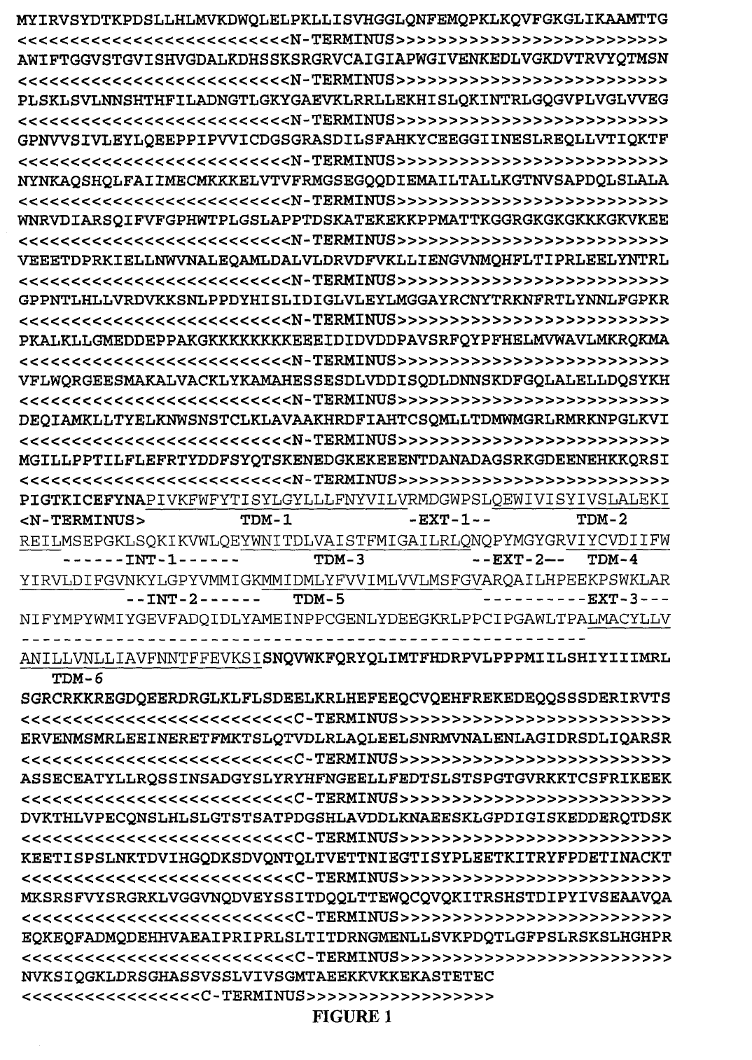

The human mlsn1 cDNA has been described in U.S. Pat. No. 5,674,739 as FOHY030. This gene is described as being expressed specifically in certain tumor cells and not in healthy tissues and was thus characterized as involved in tumor progression. The sequence of human mlsn1 cDNA is also available from Genbank (accession number AF071787). The published nucleic acid sequence encoding the open reading frame of human Mlsn1 is predicted to be 4602 nucleic acids in length (including a stop codon). This nucleotide sequence is shown in SEQ ID NO23. This published mlsn1 gene encodes a linear polypeptide of 1533 amino acids. The predicted amino acid sequence of this Mlsn1 is shown in SEQ ID NO1 and FIG. 1. The present invention stems from the identification that expression of a Mlsn1 gene product is essential for a functional capacitative calcium channel in immune-cells such as monocytes, macrophages, T-cells, B-cells and mast-cells, specifically in antigen presenting cells, more specifically in macrophages. Considering the implication of calcium influx in regulating various cell activities, this invention provides additional means in designing immuno-modulatory compounds as well as modulators compounds and in reporting immune activation and inflammation. More particularly, this invention now provides a novel target for screening immuno-modulatory compounds and deciphering immune activation gene pathways.

Therefore, a first object of the invention is the use of any purified or isolated nucleic acid encoding a human Mlsn1 or a sequence complementary thereto, wherein, the encoding nucleic acid encodes a polypeptide having the following characteristics;

(1) the N-terminal region is encoded by a consecutive sequence of at least 75 nucleotides, preferably at least 300 nucleotides, more preferably at least 750 nucleotides, most preferably 1500 nucleotides, located 5′ with respect to nucleotides encoding the transmembrane and C-terminal regions of the Mlsn1 protein. More particularly the sequence is selected within nucleotides 1 to 2268 of SEQ ID NO23,

(2) a transmembrane region encoding six membrane spanning domains, encoded by a consecutive sequence of at least 150 nucleotides, preferably at least 300 nucleotides, more preferably at least 750 nucleotides, located 3′ with respect to the nucleotides encoding the N-terminal region and 5′ with respect to nucleotides encoding the C-terminal region of Mlsn1. More particularly the sequence is selected within the nucleotides 2271 to 3045 of SEQ ID NO23, and

(3) a C-terminal region encoded by a consecutive sequence of at least 75 nucleotides, preferably at least 150 nucleotides, more preferably at least 600 nucleotides located 3′ with respect to nucleotidesencoding the N-terminal region and transmembrane region of Mlsn1. More particularly selected within nucleotides 3048 to 4599 of SEQ ID NO23, for the screening of Mlsn4 modulators, more specifically Icrac modulators. A preferred nucleic acids encoding Mlsn1 is that of SEQ ID NO23.

A further object of the invention consists of the use of any purified or isolated nucleic acid having at least 80%, preferably 90%, more preferably 95% and most preferably 98% nucleotide identity with the nucleotide sequence of SEQ ID NO23 or a sequence complementary thereto, for the screening of Mlsn1 modulators, more specifically Icrac modulators. Also encompassed by the present invention is the use of any nucleic acid that hybridizes, under stringent condition, with SEQ ID NO23, its complementary strand, or a portion thereof, and encodes a polypeptide with calcium channel activity.

A second object of the invention is the use of any purified or isolated peptide encoding a human Mlsn1 or fragment thereof, wherein, the peptide has the following characteristics;

(1) the N-terminal region comprises consecutive sequence of at least 25 amino acids, preferably at least 100 amino acids, more preferably at least 250 amino acids, most preferably 500 amino acids, located N-terminal with respect to the transmembrane and C-terminal regions of Mlsn1 protein. More particularly the sequence is selected within the amino acids 1 to 756 of SEQ ID NO1,

(2) a transmembrane region forming six membrane spanning domains, comprising a consecutive sequence of at least 50 amino acids, preferably at least 100 amino acids, more preferably at least 250 amino acids, C-terminal with respect to the N-terminal region, and N-terminal with respect to the C-terminal region of Mlsn1. More particularly the sequence is selected within amino acids 757 to 1015 of SEQ ID NO1, and

(3) a C-terminal region comprising a consecutive sequence of at least 25 amino acids, preferably at least 50 amino acids, more preferably at least 200 amino acids, located C-terminal with respect to the N-terminal and transmembrane regions of Mlsn1. More particularly the sequence is selected within amino acids 1016 to 1533 of SEQ ID NO1, for the screening of Icrac modulators, more specifically Mlsn1 modulators. Preferred amino acids encoding Mlsn1 is that of SEQ ID NO1.

A further object of the invention consists of the use of any purified or isolated peptide having at least 90%, preferably 95%, more preferably 98% and most preferably 99% amino acid identity with the sequence of SEQ ID NO1 or fragment thereof, for the screening of Mlsn1 modulators, more specifically Icrac modulators.

Another object of the invention is a purified or isolated nucleic acid sequence encoding a polypeptide, wherein said nucleic acid encodes a polypeptide having at least 90%, preferably 95%, more preferably 98% and most preferably 99% sequence identity with the polypeptide of SEQ ID NO1.

Furthermore, the discovery that Icrac function is modulated by Mlsn1 provides a means to correlate the severity of an Icrac associated disorder with the levels of expression in immune cells.

A third object of the invention is the use of any purified or isolated nucleic acid encoding a human Mlsn1 or a sequence complementary thereto, wherein, the encoding nucleic acid encodes a polypeptide having the following characteristics;

(1) the N-terminal region is encoded by a consecutive sequence of at least 75 nucleotides, preferably at least 300 nucleotides, more preferably at least 750 nucleotides, most preferably 1500 nucleotides, located 5′ with respect to nucleotides encoding the transmembrane and C-terminal regions of Mlsn1 protein. More particularly the sequence is selected within nucleotides 1 to 2268 of SEQ ID NO23,

(2) a transmembrane region encoding six membrane spanning domains, encoded by a consecutive sequence of at least 150 nucleotides, preferably at least 300 nucleotides, more preferably at least 750 nucleotides, located 3′ with respect to the nucleotides encoding the N-terminal region and 5′ with respect to nucleotides encoding the C-terminal region of Mlsn1. More particularly the sequence is selected within nucleotides 2271 to 3045 of SEQ ID NO23, and

(3) a C-terminal region encoded by a consecutive sequence of at least 75 nucleotides, preferably at least 150 nucleotides, more preferably at least 600 nucleotides, located 3′ with respect to nucleotides encoding the N-terminal region and transmembrane region of Mlsn1. More particularly the sequence is selected within nucleotides 3048 to 4599 of SEQ ID NO23, for the diagnosis of disorders associated with aberrant Icrac function in immune cells. A preferred nucleic acid encoding Mlsn1 is that of SEQ ID NO23.

A further object of the invention consists of the use of any purified or isolated nucleic acid having at least 80%, preferably 90%, more preferably 95% and most preferably 98% nucleotide identity with the nucleotide sequence of SEQ ID NO23 or a sequence complementary thereto, for the diagnosis of disorders associated with aberrant Icrac function in immune cells.

A fourth object of the invention is the use of any purified or isolated peptide encoding a human Mlsn1 or fragment thereof, wherein, the peptide has the following characteristics;

(1) the N-terminal region comprises a consecutive sequence of at least 25 amino acids, preferably at least 100 amino acids, more preferably at least 250 amino acids, most preferably 500 amino acids, located N-terminal with respect to the transmembrane and C-terminal regions of Mlsn1 protein. More particularly the sequence is selected within amino acids 1 756 of SEQ ID NO1,

(2) a transmembrane region forming six membrane spanning domains, comprising a consecutive sequence of at least 50 amino acids, preferably at least 100 amino acids, more preferably at least 250 amino acids, located C-terminal with respect to the N-terminal region and N-terminal with respect to the C-terminal region of Mlsn1. More particularly the sequence is selected within nucleic acids 757 to 1015 of SEQ ID NO1, and

(3) a C-terminal region comprising a consecutive sequence of at least 25 amino acids, preferably at least 50 amino acids, more preferably at least 200 amino acids, located C-terminal with respect to the N-terminal and transmembrane regions of Mlsn1. More particularly the sequence is selected from amino acids 1016 to 1533 of SEQ ID NO1, for the diagnosis of disorders associated with aberrant Icrac function in immune cells. A preferred nucleic acids encoding Mlsn1 is that of SEQ ID NO 1.

A further object of the invention consists of the use of any purified or isolated peptide having at least 90%, preferably 95%, more preferably 98% and most preferably 99% amino acid identity with the sequence of SEQ ID NO1 or fragment thereof, for the screening of Icrac modulators and the diagnosis of disorders associated with aberrant Icrac function in immune cells.

Structure of Mlsn1

The inventors have identified biological relevant components of the mlsn1 gene and expression products. The inventors performed gene alignment experiments with various subtypes of voltage-gated Ca²⁺ channels, using the Clustal method (MegAlign-DNAstar/Lasergene software package). In particular, the amino acid sequence of the following Trp proteins were compared, in a window of 41 residues between the predicted transmembrane segments 5 and 6 (the corresponding Genbank accession numbers are indicated): Trp, J04844; TRPL calmodulin-binding protein, M88185; TRPC7 transient receptor potential channel 7, NM 003307; Melastatin 1, AF071787 and the genome sequence of the nematode Caenorhabditis elegans, Z77132. These experiments allowed the identification of a novel calcium pore sequence in the Mlsn1 gene. The inventors believe that the active form of Mlsn1 is a membrane spanning polypeptide. This polypeptide is predicted to possess 6 transmembrane domains (TRD 1-6), and cytoplasmic N- and C-terminal domains. The N-domain, C-domain and TMDs are all connected by 5 peptide loops, two intra-cellular loops and three extra-cellular loops. Extra-cellular loop 3 forming part of the Icrac calcium pore. The TMDs incorporate the following peptides: TRD1, amino acids 757-781 (as shown in FIG. 1 of the polypeptide sequence with anotations); TMD2, amino acids 790-810 (as shown in FIG. 1); TMD3, amino acids 828-849 (as shown in FIG. 1); TMD4, amino acids 859-878 (as shown in FIG. 1); TMD5 amino acids 892-912 (as shown in FIG. 1) and TMD6, amino acids 985-1015 (as shown in FIG. 1). The N-terminal region includes amino acids 1 to 756 as shown in SEQ ID NO2 and 3, and in FIG. 1. The C-terminal region includes amino acids 1016-1533 as shown in SEQ ID NO4 and 5, and in FIG. 1. Intra-cellular loop 1 is encoded by amino acids 811-827 as shown in SEQ ID NO6 and 7, and in FIG. 1 and intra-cellular loop 2 is encoded by amino acids 879-891 as shown in SEQ ID NO8 and 9 and FIG. 1. Extra-cellular loop 1 is encoded by amino acids 782-789 as shown in SEQ ID NO10 and 11, and in FIG. 1. Extra-cellular loop 2 is encoded by amino acids 850-858 as shown in SEQ ID NO12 and 13 and in FIG. 1. Extra-cellular loop 3 is encoded by amino acids 913-984 as shown in SEQ ID NO14 to 17 and FIG. 1. The inventors believe that extra-cellular loop 3 plays a role in forming the calcium channel pore. Furthermore, the inventors believe that the 23 amino acid (amino acids 922-944) peptide sequence of SEQ ID NO16 which is predicted to be part of the calcium pore is essential for the function of Icrac.

The inventors believe that intra-cellular and extra-cellular exposed surfaces of Mlsn1 as well as the calcium pore provide vital target molecules for the development of therapeutic compounds to modulate Icrac. These surfaces include amino acids encoding the N- and C-terminal regions, as well as amino acids which encode loops connecting the transmembrane domains. Crucially, the ascribing of Icrac function to Mlsn1 has enabled the inventors to identify a calcium pore within the transmembrane region, and an external peptide loop. This loop is thought to directly modulate calcium flux in Icrac. Both the calcium pore and associated loop hold special significance in the development of therapeutics for the treatment of aberrant Icrac function. Furthermore, peptides of Mlsn1 allow the development of diagnostic tools to assay for Icrac function and levels in immune cells.

Likewise the nucleic acids which encode the said peptides offer opportunities to identify modulators of Icrac, and the development of diagnostic tools.

Peptides of Mlsn1

The inventors believe that the N-terminal cytoplasmic region of Mlsn1 could play a role in the translocation of the Mlsn1 polypeptide from the cytoplasm to the cell membrane.

Therefore, an object of the invention is a purified or isolated human Mlsn1 N-terminal region, wherein the N-terminal region comprises a consecutive sequence of at least 25 amino acids, preferably at least 100 amino acids, more preferably at least 250 amino acids and most preferably at least 500 amino acids, located N-terminal of the first transmembrane region of the Mlsn1 protein. Preferred is a consecutive sequence taken from the amino acid sequences of SEQ ID NO2 or 3. More preferred are the sequences of SEQ ID NO2 or 3.

A further object of the invention resides in a purified or isolated human Mlsn1 N-terminal region, wherein the N-terminal region has at least 90%, preferably 95%, more preferably 98% and most preferably 99% amino acid identity with any of the peptides of amino acid sequences SEQ ID NO2 or 3, or fragments thereof.

The inventors also believe that the C-terminal region of Mlsn1 plays a role in the modulation of activity of Mlsn1. Therefore, a second object of the invention consists of a purified or isolated human Mlsn1 C-terminal region, wherein the C-terminal region comprises a consecutive sequence of at least 25 amino acids, preferably at least 50 amino acids, more preferably at least 100 amino acids and most preferably at least 200 amino acids, located C-terminal of the sixth transmembrane region of the Mlsn1 protein. Preferred is a consecutive sequence taken from the amino acid sequence of SEQ ID NO4 or 5. More preferred is the sequences of SEQ ID NO4 or 5.

A further object of the invention resides in a purified or isolated human Mlsn1 C-terminal region, wherein the C-terminal region has at least 90%, preferably 95%, more preferably 98% and most preferably 99% amino acid identity with any of the peptides of amino acid sequences SEQ ID NO4 or 5, or fragments thereof.

The inventors believe that peptide encoding intra-cellular loop-1 of Mlsn1 play an important role in the regulation of Mlsn1 and its capacity to transduce intra-cellular signals. Therefore, a third object of the invention is a purified or isolated human Mlsn1 intra-cellular loop-i region, wherein the intra-cellular loop-1 region comprises a consecutive sequence of at least 7 amino acids, preferably at least 10 amino acids, more preferably at least 15 amino acids, located C-terminal of the second transmembrane region and N-terminal to the third transmembrane region of the Mlsn1 protein. Preferred is a consecutive sequence taken from the amino acid sequence of SEQ ID NO6 or 7. More preferred are the sequences of SEQ ID NO6 or 7.

A further object of the invention resides in a purified or isolated human Mlsn1 intra-cellular loop-1 region, wherein the intra-cellular loop-1 region has at least 90%, preferably 95%, more preferably 98% and most preferably 99% amino acid identity with any of the peptides of amino acid sequences SEQ ID NO6 or 7, or fragments thereof.

The inventors believe that peptide encoding intra-cellular loop-2 of Mlsn1 also play an important role in the regulation of Mlsn1 and its capacity to transduce intra-cellular signals. Therefore, a fourth object of the invention is a purified or isolated human Mlsn1 intra-cellular loop-2 region, wherein the intra-cellular loop-2 region comprises a consecutive sequence of at least 7 amino acids, preferably at least 10 amino acids, located C-terminal of the fourth transmembrane region and N-terminal of the fifth transmembrane region of the Mlsn1 protein. Preferred is a consecutive sequence taken from the amino acid sequences of SEQ ID NO8 or 9. More preferred is the sequences of SEQ ID NO8 or 9.

A further object of the invention is a purified or isolated human Mlsn1 intra-cellular loop-2 region, wherein the intra-cellular loop-2 region has at least 90%, preferably 95%, more preferably 98% and most preferably 99% amino acid identity with any of the peptides of amino acid sequences SEQ ID NO8 or 9, or fragments thereof.

The inventors believe that the peptides encoding extra-cellular loop 1 of Mlsn1 may play a crucial role in the modulation of calcium through the Icrac channel. Therefore a fifth object of the invention is purified or isolated human Mlsn1 extra-cellular loop-1 region, wherein the extra-cellular loop-1 region comprises a consecutive sequence of at least 5 amino acid located C-terminal of the first transmembrane region and N-terminal of the second transmembrane region of the Mlsn1 protein. Preferred is a consecutive sequence taken from the amino acid sequences of SEQ ID NO10 or 11. More preferred is the sequence of SEQ ID NO10 or 11.

A further object of the invention is purified or isolated human Mlsn1 extra-cellular loop-1 region, wherein the extra-cellular loop-1 region has at least 90%, preferably 95%, more preferably 98% and most preferably 99% amino acid identity with any of the peptides of amino acid sequences SEQ ID NO10 or 11, or fragments thereof.

The inventors believe that the peptides encoding extra-cellular loop 2 region of Mlsn1 may also play a crucial role in the modulation of calcium through the Icrac channel. Therefore, a sixth object of the invention is purified or isolated human Mlsn1 extra-cellular loop-2 region, wherein the extra-cellular loop-2 region comprises a consecutive sequence of at least 5 amino acids, located C-terminal of the third transmembrane region and N-terminal of the fourth transmembrane region of the Mlsn1 protein. Preferred is a consecutive sequence taken from the amino acid sequence of SEQ ID NO12 or 13. More preferred is the sequence of SEQ ID NO12 or 13.

A further object is purified or isolated human Mlsni extra-cellular loop-2 region, wherein the extra-cellular loop-2 region has at least 90%, preferably 95%, more preferably 98% and most preferably 99% amino acid identity with any of the peptides of amino acid sequences SEQ ID NO12 or 13, or fragments thereof.

The inventors believe that the peptides encoding extra-cellular loop-3 region, has a function in the modulation of calcium flux in Icrac. Therefore, a seventh object of the invention is a purified or isolated human Mlsn1 extra-cellular loop-3 region, wherein the extra-cellular loop-3 region comprises a consecutive sequence of at least 7 amino acids, preferably at least 10 amino acids, more preferably at least 20 amino acids, most preferably at least 50 amino acids, located C-terminal of the fifth transmembrane region and N-terminal of the sixth transmembrane region of the Mlsn1 protein. Preferred is a consecutive sequence taken from the amino acid sequence of SEQ ID NO14, 15, 16 or 17. More preferred are the sequences of SEQ ID NO14, 15, 16 or 17.

A further object of the invention is purified or isolated human Mlsn1 extra-cellular loop-3 region, wherein the extra-cellular loop-3 region has at least 90%, preferably 95%, more preferably 98% and most preferably 99% amino acid identity with any of the peptides of amino acid sequences SEQ ID NO14, 15, 16 or 17, or fragments thereof.

The inventors believe that peptides which contain both intra- and extra-cellular loop regions and transmembrane regions but lacking associated N- and C-terminal regions could play a role in the understanding of Icrac and the development of modulators of Icrac and diagnostic tools. Therefore, an eighth object of the invention is purified or isolated human Mlsn1 peptide containing both intra- and extra-cellular loop regions, wherein said intra- and extra-cellular loop region comprises a consecutive sequence of at least 50 amino acids, preferably at least 100 amino acids and most preferably at least 250 amino acids, located C-terminal to the N-terminal region and N-terminal to the C-terminal region of the Mlsn1 protein. Preferred is a consecutive sequence taken from the amino acid sequence of SEQ ID NO18 or 19. More preferred is the sequence of SEQ ID NO18 or 19.

A further object of the invention is purified or isolated human Mlsn1 peptide containing both intra- and extra-cellular loop regions, wherein the intra- and extra-cellular loop region has at least 90%, preferably 95%, more preferably 98% and most preferably 99% amino acid identity with any of the peptides of amino acid sequences SEQ ID NO18 or 19, or fragments thereof.

Amino acid identity may be determined using various methods known in the art. In particular, amino acid identity (or sequence homology) may be assessed by commercially available computer programs, such as CLUSTAL or BLAST (NCBI). Various search or alignment parameters may also be used. In a preferred embodiment, % amino acid (or nucleic acid) identity is determined using the CLUSTAL-W program (Compugen).

Nucleic Acids Encoding Peptides of Mlsn1

This invention also relates generally to the use of nucleic acid sequences encoding said Mlsn1 peptides for the development of screening and diagnostic technologies.

An object of the invention is a purified or isolated nucleic acid sequence encoding the human Mlsn1 N-terminal region, wherein the N-terminal region is encoded by a consecutive sequence of at least 75 nucleotides, preferably at least 300 nucleotides, more preferably at least 750 nucleotides and most preferably at least 1500 nucleotides, located 5′ with respect to nucleotides encoding the first transmembrane region of the Mlsn1 protein. Preferred is a consecutive sequence taken from the nucleic acid sequences of SEQ ID NO24 or 25. More preferred is the sequence of SEQ ID NO24 or 25.

A further object of the invention is a purified or isolated nucleic acid sequence encoding the human Mlsn1 N-terminal region, wherein the nucleic acid has at least 80%, preferably 90%, more preferably 95% and most preferably 98% nucleic acid identity with any of the nucleotide sequences or SEQ ID NO24 or 25, or a complementary sequences thereto.

Another object of the invention is a purified or isolated nucleic acid sequence encoding a polypeptide, wherein said nucleic acid encodes a polypeptide having at least 90%, preferably, 95, more preferably 98% and most preferably 99% with any of the polypeptides of SEQ ID NO2 or 3.

A further object of the invention is a purified or isolated nucleic acid sequence encoding the human Mlsn1 C-terminal region, wherein the C-terminal region is encoded by a consecutive sequence of at least 75 nucleotides, preferably at least 150 nucleotides, more preferably at least 300 nucleotides and most preferably at least 600 nucleotides, located 5′ with respect to nucleotides encoding the sixth transmembrane region of the Mlsn1 protein. Preferred is a consecutive sequences taken from the nucleic acid sequence of SEQ ID NO26 or 27. More preferred is the sequence of SEQ ID NO26 or 27.

A further object of the invention is a purified or isolated nucleic acid sequence encoding the human Mlsn1 C-terminal region, wherein the nucleic acid has at least 80%, preferably 90%, more preferably 95% and most preferably 98% nucleic acid identity with any of the nucleotide sequences or SEQ ID NO26 or 27, or a complementary sequences thereto.

A further object of the invention is a purified or isolated nucleic acid sequence encoding a polypeptide, wherein said nucleic acid encodes a polypeptide having at least 90%, preferably, 95%, more preferably 98% and most preferably 99% with any of the polypeptides of SEQ ID NO4 or 5.

A further object of the invention resides is a purified or isolated nucleic acid sequence encoding the human Mlsn1 intra-cellular loop-1 region, wherein the intra-cellular loop-1 region is encoded by a consecutive sequence of at least 21 nucleotides, preferably at least 30 nucleotides, more preferably at least 45 nucleotides, located 3′ with respect to nucleotides encoding the second transmembrane region and 5′ with respect to nucleotides encoding the third transmembrane region of the Mlsn1 protein. Preferred is a consecutive sequence taken from the nucleic acid sequence of SEQ ID NO28 or 29. More preferred is the sequence of SEQ ID NO28 or 29.

A further object of the invention is a purified or isolated nucleic acid sequence encoding the human Mlsn1 loop-1 region, wherein the nucleic acid has at least 80%, preferably 90%, more preferably 95% and most preferably 98% nucleic acid identity with any of the nucleotide sequences or SEQ ID NO28 or 29, or a complementary sequences thereto.

Another object of the invention is a purified or isolated nucleic acid sequence encoding a polypeptide, wherein said nucleic acid encodes a polypeptide having at least 90%, preferably, 95%, more preferably 98% and most preferably 99% with any of the polypeptides of SEQ ID NO6 or 7.

A further object of the invention is a purified or isolated nucleic acid sequence encoding the human Mlsn1 intra-cellular loop-2 region, wherein the intra-cellular loops-2 region is encoded by a consecutive sequence of at least 21 nucleotides, preferably at least 30 nucleotides, located 3′ with respect to nucleotides encoding the fourth transmembrane region and 5′ with respect to nucleotides encoding the fifth transmembrane region of the Mlsn1 protein. Preferred is a consecutive sequence taken from the nucleic acid sequence of SEQ ID NO30 or 31. More preferred is the sequence of SEQ ID NO30 or 31.

A further object of the invention is a purified or isolated nucleic acid sequence encoding the human Mlsn1 intra-cellular loop-2 region, wherein the nucleic acid has at least 80%, preferably 90%, more preferably 95% and most preferably 98% nucleic acid identity with any of the nucleotide sequences or SEQ ID NO30 or 31, or a complementary sequences thereto.

Another object of the invention is a purified or isolated nucleic acid sequence encoding a polypeptide, wherein said nucleic acid encodes a polypeptide having at least 90%, preferably, 95%, more preferably 98% and most preferably 99% with any of the polypeptides of SEQ ID NO8 or 9.

A further object of the invention is a purified or isolated nucleic acid sequence encoding the human Mlsn1 extra-cellular loop-1 region, wherein the extra-cellular loops-1 region is encoded by a consecutive sequence of at least 15 nucleotides, located 3′ with respect to nucleotides encoding the first transmembrane region and 5′ with respect to nucleotides encoding the second transmembrane region of the Mlsn1 protein. Preferred is a consecutive sequence taken from the nucleic acid sequence of SEQ ID NO32 or 33. More preferred is the sequence of SEQ ID NO32 or 33.

A further object of the invention is purified or isolated nucleic acid sequence encoding the human Mlsn1 extra-cellular loop-1 region, wherein the nucleic acid has at least 80%, preferably 90%, more preferably 95% and most preferably 98% nucleic acid identity with any of the nucleotide sequences or SEQ ID NO32 or 33, or a complementary sequences thereto.

Another object of the invention is a purified or isolated nucleic acid sequence encoding a polypeptide, wherein said nucleic acid encodes a polypeptide having at least 90%, preferably, 95%, more preferably 98% and most preferably 99% with any of the polypeptides of SEQ ID NO10 or 11.

A further object of the invention is purified or isolated nucleic acid sequence encoding the human Mlsn1 extra-cellular loop-2 region, wherein the extra-cellular loops-2 region is encoded by a consecutive sequence of at least 15 nucleotides, located 3′ with respect to nucleotides encoding the third transmembrane region and 5′ with respect to nucleotides encoding the fourth transmembrane region of the Mlsn1 protein. Preferred is a consecutive sequence taken from the nucleic acid sequence of SEQ ID NO34 or 35. More preferred is the sequence of SEQ ID NO34 or 35.

A further object of the invention is purified or isolated nucleic acid sequence encoding the human Mlsn1 extra-cellular loop-2 region, wherein the nucleic acid has at least 80%, preferably 90%, more preferably 95% and most preferably 98% nucleic acid identity with any of the nucleotide sequences or SEQ ID NO34 or 35, or a complementary sequences thereto.

Another object of the invention is a purified or isolated nucleic acid sequence encoding a polypeptide, wherein said nucleic acid encodes a polypeptide having at least 90%, preferably, 95%, more preferably 98% and most preferably 99% with any of the polypeptides of SEQ ID NO12 or 13.

A further object of the invention is purified or isolated nucleic acid sequence encoding the human Mlsn1 extra-cellular loop-3 region, wherein the extra-cellular loops-3 region is encoded by a consecutive sequence of at least 21 nucleotides, preferably at least 30 nucleotides, more preferably at least 60 nucleotides and most preferably at least 150 nucleotides 3′ with respect to nucleotides encoding the fifth transmembrane region and 5′ with respect to nucleotides encoding the sixth transmembrane region of the Mlsn1 protein. Preferred is a consecutive sequence taken from the nucleic acid sequence of SEQ ID NO36, 37 or 38. More preferred is the sequence of SEQ ID NO36, 37 or 38.

A further object is purified or isolated nucleic acid sequence encoding the human Mlsn1 extra-cellular loop-3 region, wherein the nucleic acid has at least 80%, preferably 90%, more preferably 95% and most preferably 98% nucleic acid identity with any of the nucleotide sequences or SEQ ID NO36, 37 or 38, or a complementary sequences thereto.

Another object of the invention is a purified or isolated nucleic acid sequence encoding a polypeptide, wherein said nucleic acid encodes a polypeptide having at least 90%, preferably, 95%, more preferably 98% and most preferably 99% with any of the polypeptides of SEQ ID NO14, 15, 16 or 17.

An other object of the invention is purified or isolated nucleic acid sequence encoding the human Mlsn1 peptide containing both intra- and extra-cellular loop regions, wherein said intra- and extra-cellular loop region is encoded by a consecutive sequence of at least 150 nucleotides, preferably at least 300 nucleotides and most preferably at least 750 nucleotides 3′ with respect to nucleotides encoding the N-terminal region and 5′ with respect to nucleotides encoding the C-terminal regions of the Mlsn1 protein. Preferred is a consecutive sequence taken from the nucleic acid sequence of SEQ ID NO39 or 40. More preferred is the sequence of SEQ ID NO39 or 40.

Another object of the invention is purified or isolated nucleic acid sequence encoding the human Mlsn1 peptide containing both intra- and extra-cellular loop regions, wherein the nucleic acid has at least 80%, preferably 90%, more preferably 95% and most preferably 98% nucleic acid identity with any of the nucleotide sequences or SEQ ID NO39 or 40, or a complementary sequences thereto.

Another object of the invention is a purified or isolated nucleic acid sequence encoding a polypeptide, wherein said nucleic acid encodes a polypeptide having at least 90%, preferably, 95%, more preferably 98% and most preferably 99% with any of the polypeptides of SEQ ID NO18 or 19.

Nucleic acid sequence identity may be determined using various methods known in the art. In particular, nucleic acid sequence identity (or sequence homology) may be assessed by commercially available computer programs, such as CLUSTAL or BLAST (NCBI). Various search or alignment parameters may also be used. In a preferred embodiment, % amino acid (or nucleic acid) identity is determined using the CLUSTAL-W program (Compugen).

Transcription Promoters of Mlsn1

The inventors have identified three genomic nucleic acid sequences 5′ to the published codon encoding Methionine 1 of the Mlsn1 gene that encode transcription promoter sequences. The inventors believe that nucleic acids encoding transcription promoter(s) elements of Mlsn1 are targets for the development of therapeutics which modulate Icrac activity. A first preferred nucleic acid sequence is a nucleic acid sequence which encodes an Mlsn1 transcription promoter of SEQ ID NO41 or a complementary sequence thereto. A further object of the invention consists of nucleic acid sequences having at least 80%, preferably 90%, more preferably 95% and most preferably 98% nucleotide identity with the nucleotide sequence of SEQ ID NO41 or a complementary sequence thereto.

A second preferred nucleic acid sequence is a nucleic acid sequence which encodes an Mlsn1 transcription promoter of SEQ ID NO42 or a complementary sequence thereto. A further object of the invention consists of nucleic acid sequences having at least 80%, preferably 90%, more preferably 95% and most preferably 98% nucleotide identity with the nucleotide sequence of SEQ ID NO42 or a complementary sequence thereto.

A third preferred nucleic acid sequence is a nucleic acid sequence which encodes an Mlsn1 transcription promoter of SEQ ID NO43 or a complementary sequence thereto. A further object of the invention consists of nucleic acid sequence having at least 80%, preferably 90%, more preferably 95% and most preferably 98% nucleotide identity with the nucleotide sequence of SEQ ID NO43 or a complementary sequence thereto.

Expression of Mlsn1

The inventors discovered that the Mlsn1 gene was expressed in U937 cells differentiated into a macrophage phenotype, and that the product of this gene functions as a calcium channel, more specifically as an Icrac channel. Mlsn1 -specific primers were designed and used to screen for Mlsn1 gene expression in various cells. The composition of these primers are indicated in SEQ ID NO44 to 47. With said primer pairs, Mlsn1-specific PCR products were obtained from U937 cells. Furthermore, cDNA subtraction experiments were performed using DNA preparations from differentiated and undifferentiated U937 cells. The results obtained demonstrate that the expression of the Mlsn1 gene product in U937 cells is differentially increased in dbcAMP-differentiated U937 cells (macrophage-like) as compared to undifferentiated U937-cells (monocyte like). The upregulation of the mlsn1 gene correlates with the expression of the Icrac phenotype in U937 cells (18) and thus establishes that expression upregulation of Mlsn1 in these cells coincides with the increased Icrac phenotype. Sequence specific evidence for Mlsn1 involvement in the Icrac phenotype was obtained by the demonstration that Mlsn1 -specific anti-sense treatment of U937 cells during dbcAMP induced differentiation could significantly decrease the calcium signal in these cells.

Accordingly, this invention unexpectedly demonstrates that Mlsn1 is expressed in immune cells such as macrophages and that the down-regulation of the Mlsn1 gene expression during differentiation of U937 cells to macrophage like cells decreases calcium influx in these cells. This invention also demonstrates that Mlsn1 gene product expression is up-regulated during U937 cell differentiation to macrophage like cells, which correlates with the maturation stage of these cells and their ability to exhibit the Icrac phenotype.

Production of Mlsn1 Polypeptides or Nucleic Acids

Recombinant Expression Vecotors

The present invention also encompasses a family of recombinant vectors comprising any one of the nucleic acids described herein. Thus, the invention further deals with a recombinant vector comprising a nucleic acid selected from the group consisting of:

(a) a purified or isolated nucleic acid encoding Mlsn1, preferably human Mlsn1 or Mlsn1 transcription promoter element(s), and more preferably a polypeptide having at least 90% amino acid identity with a polypeptide selected from the group consisting of; nucleic acids encoding Mlsn1, such as SEQ ID NO1, nucleic acids encoding the N-terminal region of Mlsn1, such as SEQ ID NO2 and 3, nucleic acids encoding C-terminal region of Mlsn1, such as SEQ ID NO4 and 5, nucleic acids encoding Intra-cellular loops of Mlsn1, such as SEQ ID NO6 to 9, nucleic acids encoding extra-cellular loops 1 and 2 of Mlsn1, such as SEQ ID NO10 to 13, nucleic acids encoding extra-cellular loop 3 of Mlsn1, such as SEQ ID NO14 to 17, nucleic acids encoding both intra- and extra cellular loops of Mlsn1, such as SEQ ID NO18 and 19 and nucleic acids encoding transcription promoter elements of SEQ ID NO41 to 43, or a sequence complementary thereto;

(b) a purified or isolated nucleic acid having at least 80% nucleotide identity with a polynucleotide selected from the group consisting of the nucleotide sequences of SEQ ID NO23 to43, or a sequence complementary thereto;

(c) a purified or isolated polynucleotide comprising at least 10 consecutive nucleotides of a nucleic acid described in (a) or (b), or a sequence complementary thereto.

In a first preferred embodiment a recombinant vector of the invention is used to amplify the inserted polynucleotide derived from the nucleic acid encoding a Mlsn1 of the invention or Mlsn1 transcription promoter in a suitable host cell, this polynucleotide being amplified every time the recombinant vector replicates.

A second preferred embodiment of the recombinant vectors according to the invention consists of expression vectors comprising a nucleic acid encoding an Mlsn1 of the invention, preferably a nucleic acid encoding a human Mlsn1, and more preferably a nucleic acid encoding a polypeptide selected from the group consisting of the amino acid sequences of SEQ ID No 1 to 22.

Recombinant expression vectors comprising a nucleic acid encoding the peptide fragments of an Mlsn1 that are specified in the present specification are also part of the invention.

Within certain embodiments, expression vectors can be employed to express Mlsn1 of the invention or a peptide fragment thereof which can then be purified and for example, be used as a immunogen in order to raise specific antibodies directed against said Mlsn1 protein or a peptide fragment thereof.

In another embodiment, the expression vectors are used for constructing transgenic animals and also for gene therapy, notably for anti-sense therapy.

Expression requires that appropriate signals are provided in the vectors, said signals including various regulatory elements such as enhancers/promoters from both viral and mammalian sources that drive expression of the genes of interest in host cells. The regulatory sequences of the expression vectors of the invention are operably linked to the nucleic acid encoding the Mlsn1 protein of interest or a peptide fragment thereof.

As used herein, the term “operably linked” refers to a linkage of polynucleotide elements in a functional relationship. For instance, a promoter or an enhancer is operably linked to a coding sequence if it affects the transcription of the coding sequence.

More precisely, two DNA molecules (such as a polynucleotide containing a promoter region and a polynucleotide encoding a desired polypeptide or polynucleotide) are said to be “operably linked” if the nature of the linkage between the two polynucleotides does not: (1) result in the introduction of a frame-shift mutation or (2) interfere with the ability of the polynucleotide containing the promoter to direct the transcription of the coding polynucleotide.

Generally, recombinant expression vectors will include origins of replication, selectable markers, permitting transformation of the host cell, and a promoter derived from a highly expressed gene to direct transcription of a downstream structural sequence. The heterologous structural sequence is assembled in an appropriate frame with the translation, initiation and termination sequences, and preferably a leader sequence capable of directing sequences of the translated protein into the periplasmic space or the extra-cellular medium.

In a specific embodiment wherein the vector is adapted for transfecting and expressing desired sequences in mammalian host cells, preferred vectors will comprise an origin of replication from the desired host, a suitable promoter and an enhancer, and also any necessary ribosome binding sites, polyadenylation site, transcriptional termination sequences, and optionally 5′-flanking non-transcribed sequences.

DNA sequences derived from the SV 40 viral genome, for example SV 40 origin, early promoter, enhancer, and polyadenylation sites may be used to provide the required non-transcribed genetic elements.

Additionally, a recombinant expression vector of the invention advantageously also comprises an untranscribed polynucleotide located at the 3′ end of the coding sequence (ORF), this 3′-UTR polynucleotide being useful for stabilizing the corresponding MRNA or for increasing the expression rate of the vector insert if this 3′-UTR harbors regulation signal elements such as enhancer sequences.

Suitable promoter regions used in the expression vectors according to the invention are chosen taking into account the host cell in which the heterologous nucleic acids have to be expressed.

A suitable promoter may be heterologous with respect to the nucleic acid for which it controls the expression, or alternatively can be endogenous to the native polynucleotide containing the coding sequence to be expressed.

Additionally, the promoter is generally heterologous with respect to the recombinant vector sequences within which the construct promoter/coding sequence has been inserted.

Preferred bacterial promoters are the Lacd, LacZ, T3 or T7 bacteriophage RNA polymerase promoters, the lambda PR, PL and trp promoters (a EP-0 036 776), the polyhedrin promoter, or the p10 protein promoter from baculovirus (kit Novagen; Smith et al., (1983); O'Reilly et al. (1992).

Preferred selectable marker genes contained in the expression recombinant vectors of the invention for selection of transformed host cells are preferably dehydrofolate reductase or neomycin resistance for eukaryotic cell culture, TRP1 for S. cerevisiae or tetracycline, rifampicin or ampicillin resistance in E. coli, or Levamsaccharase for Mycobacteria, this latter marker being a negative selection marker.

Preferred bacterial vectors of the invention are listed hereafter as illustrative but not limitative examples:

pQE70, pQE60, pQE-9 (Quiagen), pD10, phagescript, psiX174, p.Bluescript SK, pNH8A, pNH16A, pNH18A, pNH46A (Stratagene); pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (QIA express).

Preferred bacteriophage recombinant vectors of the invention are P1 bacteriophage vectors such as described by Sternberg N. L. (1992; 1994).

A suitable vector for the expression of Mlsn1 polypeptide of the invention or a fragment thereof, is a baculovirus vector that can be propagated in insect cells and in insect cell-lines. A specific suitable host vector system is the pVL 1392/1393 baculovirus transfer vector (Pharmingen) that is used to transfect the SF9 cell line (ATCC NOCRL 1711) which is derived from spodoptera frugiperda.

The recombinant expression vectors from the invention may also be derived from an adenovirus such as those described by Feldman and Steig. (1996) or Ohno et al. (1994).

Another preferred recombinant adenovirus according to this specific embodiment of the present invention is the human adenovirus type two or five (Ad 2 or Ad 5) or an adenovirus of animal origin (French Pat. Application no. FR 93 05 954).

Particularly preferred retrovirus as for the preparation or construction of retroviral in vitro or in vivo gene delivery vehicles of the present invention include retroviruses selected from the group consisting of Mink-Cell Focus Inducing Virus, murine sarcoma virus, and Ross Sarcoma Virus. Other preferred retroviral vectors are those described in Roth et al. (1996), in PCT Application WO 93/25 234, in PCT Application WO 94/06920, and also in Roux et al. (1989), Julan et al.(1992) and Nada et al. (1991).

Yet, another viral vector system that is contemplated by the invention consist in the adeno associated viruses (AAV) such as those described by Flotte et al. (1992), Samulski et al. (1989) and McLaughlin et al. (1996).

Thus, a further object of the invention consists of a recombinant expression vector comprising a nucleic acid encoding an Mlsn1 or a peptide fragment thereof or a variant thereof, wherein said nucleic acid is operably linked to a promoter sequence.

In a preferred embodiment, this nucleic acid encodes a human Mlsn1, a fragment of human Mlsn1, or Mlsn1 transcription promoter, and preferably that of SEQ ID NO23 and nucleic acid sequence encoding peptide sequence of SEQ ID NO1, or a variant or a peptide fragment thereof. Preferred fragments include those of SEQ ID NO 2-22 and 24 to 40. Preferred Mlsn1 transcription promoters include those of SEQ ID NO41 to 43.

In a specific embodiment of this invention, any of the identified Mlsn1 promoter elements of SEQ ID NO41 to 43 could be used to modulate the expression of a reporter molecule, as part of an expression construct. Furthermore, these Mlsn1 transcription promoter elements could be used to regulate Mlsn1 gene of gene fragment expression as part of a construct.

Host Cells Expressing Mlsn1

Host cells that endogenously express Mlsn1 or have been transformed or transfected with one of the nucleic acids described herein, or with one of the recombinant vector, particularly recombinant expression vector, described herein are also part of the present invention.

Also included are host cells that are transformed (prokaryotic cells) or are transfected (eukaryotic cells) with a recombinant vector such as one of those described above. Preferred host cells used as recipients for the expression vectors of the invention are the following:

(a) prokaryotic host cells: Escherichia coli, strains. (i.e. DH5-α, strain) Bacillus subtilis, Salmonella typhimurium and strains from species like Pseudomonas, Streptomyces and Staphylococcus;

(b) eukaryotic host cells: T-cell lines (ECACC U937, 85011440; ECACC J.CaM1.6, 96060401; ECACC Jurkat E6.1, 88042803 and ECACC J45.01, 93031145), HeLa cells (ATCC NOCCL2; NOCCL2.1; NOCCL2.2), Cv 1 cells (ATCC NOCCL70), COS cells (ATCC NOCRL 1650; NOCRL 1651), Sf-9 cells (ATCC NOCRL 1711), C127 cells (ATCC NOCRL-1804), 3T3 cells (ATCC NOCRL-6361), CHO cells (ATCC NOCCL-61), human kidney 293 cells (ATCC NO 45504; NOCRL-1573), BHK (ECACC NO84100 501; NO84111301), PC12 (ATCC NO CRL-1721), NT2, SHSY5Y (ATCC NO CRL-2266), NG108 (ECACC NO88112302) and F11, SK-N-SH (ATCC NO CRL-HTB-11), SK-N-BE(2) (ATCC NO CRL-2271), IMR-32 (ATCC NO CCL-127). A preferred system to which the gene of the invention can be expressed are cell lines such as T-cell lines, B-cell lines, mast cell lines, jurkat cell lines, U937 cell lines, KU-812 cell lines, COS cells, 3T3 cells, HeLa cells, 292 cells and CHO cells. A most preferred system for the efficient expression of Mlsn1 involves the use of T-cell lines. The gene can be expressed through an endogenous promoter of native T-cells, or through an exogenous promoter. Suitable exogenous promoters include such as SV40 and CMV, or perhaps a eukaryotic promoter such as the tetracycline promoter. The preferred promoter when Mlsn1 is endogenously expressed is an endogenous promoter. A prefered promoter in a recombinant cell line would be CMV.

In a specific embodiment of the host cells described above, these host cells have also been transfected or transformed with a polynucleotide or a recombinant vector allowing the expression of a natural ligand of Mlsn1 or a modulator of Mlsn1.

The present invention also concerns a method for producing one of the Mlsn1 polypeptides or peptides described herein and especially a polypeptide selected from the group consisting the amino acid sequences of SEQ ID NO1 to 22, wherein said method comprises the steps of:

(a) inserting the nucleic acid encoding the desired Mlsn1 polypeptide or peptide fragment thereof in an appropriate vector;

(b) culturing, in an appropriate culture medium, a host cell previously transformed or transfected with the recombinant vector of step (a);

(c) harvesting the culture medium thus conditioned or lyse the host cell, for example by sonication or by an osmotic shock;

(d) separating or purifying, from said culture medium, or from the pellet of the resultant host cell lysate, the thus produced Mlsn1 polypeptide of interest.

In a first preferred embodiment of the above method, the nucleic acid to be inserted in the appropriate vector has previously undergone an amplification reaction, using a pair of primers.

Preferred primers used for such an amplification reaction are primer pairs which amplify nucleic acids encoding the Mlsn1 open reading frame and fragments thereof.

In a second preferred embodiment of the above method, the polypeptide thus produced is further characterized, for example by binding onto an immuno-affinity chromatography column on which polyclonal or monoclonal antibodies directed to Mlsn1 polypeptide or a peptide fragment thereof, have previously been immobilised.

Purification of the recombinant Mlsn1 proteins according to the present invention or a peptide fragment thereof may be carried out by passage onto a nickel or copper affinity chromatography column.

In another embodiment, the Mlsn1 polypeptides or peptide fragments thus obtained may be purified, for example, by high performance liquid chromatography, such as reverse phase and/or cationic exchange HPLC, as described by Rougeot et al. (1994).

The reason to prefer this kind of peptide or protein purification is the lack of by-products formed in the elution samples which renders the resultant purified protein or peptide more suitable for therapeutic use.

Production of Mlsn1 or a Fragment Thereof

The Mlsn1 protein or fragments thereof can be prepared using recombinant technology, cell lines or chemical synthesis. Recombinant technology and chemical synthesis of the Mlsn1 or fragments thereof can allow the modification of the gene encoding the Mlsn1 to include such features as recognition tags, cleavage sites and modifications of the Mlsn1 polypeptide or fragments thereof. For efficient polypeptide production, the endogenous expression system or recombinant expression system should allow the Mlsn1 polypeptide to be expressed at and transported to the cell surface in a functional form or allow production of Mlsn1 fragments which can be purified. Preferred cell lines are those which allow high levels of expression of Mlsn1 or fragments thereof. Such cell lines include cell lines which naturally expresss Mlsn1 or common mammalian cell lines expressing Mlsn1 such as CHO cells and COS cells, etc., or more specific immune cell lines such as T-cell lines. However, other cell types which are commonly used for recombinant protein production such as insect cells, amphibian cells such as oocytes, yeast and procaryotic cell lines such as E.coli can also be considered.

The Mlsn1 or fragments thereof can be utilised in a ligand screen either as a purified protein, as a protein chimera such as those described in phage display, as a cell membrane (lipid or detergent) preparation, or in intact cells.

The Mlsn1 polypeptide or fragment thereof can be utilised in a functional screen format or ligand binding screen format. Examples of both screening formats are provided below.

Screening for Mlsn1 Ligands

The present invention also concerns methods for screening ligand substances or molecules that are able to modulate the biological activity of the Mlsn1 gene or gene product of the invention. For example diseases where there is a down-regulation of the immune system and conditions associated with virus and micro-organism infection, enhancement of the activity of Mlsn1 would be beneficial. In conditions such as asthma, inflammation and auto-immune disease it would be beneficial to identify ligands which can down-regulate the activity of the Mlsn1 gene.

In this regard, this invention now discloses the use of a Mlsn1 gene product for the screening of compound(s) that modulate the activity of immune cells, in particular antigen presenting cells, more specifically for the screening of compound(s) that modulate the activity of macrophages, in vitro, ex vivo or in vivo.

This invention also discloses the use of Mlsn1 promoter elements for the screening of compounds that modulate the activity of immune cells, in particular antigen presenting cells, more specifically for the screening of compounds that modulate the activity of macrophages, in vitro, ex vivo or in vivo.

Several screening assays can be used within the instant invention, such as binding assays or functional assays. In a particular embodiment, this invention more specifically relates to methods of screening for immuno-modulatory compounds, comprising;

a) contacting a test compound with Mlsn1 gene, an Mlsn1 gene product or Mlsn1 promoter element and,

b) measuring the ability of said test compound to interact with the Mlsn1 gene, Mlsn1 gene product or transcription promoter element (i.e., binding assay).

In another embodiment, the screening method comprises;

a) contacting a test compound with an Mlsn1 expressing cell, more particularly an antigen presenting cell (or a macrophage) and,

b) determining the ability of said test compound to affect Mlsn1 gene or an Mlsn1 gene product activity within the cells.

In a further embodiment, the screening method comprises contacting a test compound with a cell expressing a reporter gene under the control of Mlsn1 transcription promoter elements, more particularly an antigen presenting cell (or a macrophage), expressing the said promoter/reporter construct and determining the ability of the said test compound to modulate reporter expression.

Ligand binding screens to the Mlsn1 gene, polypeptide, a fragment thereof or Mlsn1 transcription promoter element can allow rapid identification of ligands which are capable of interacting with the gene or product(s). Further testing may be required to determine the therapeutic activity of these ligands. Development of a functional screen, whereby the activity of Mlsn1 is measured (or reporter activity measured) upon ligand binding allows ligands to be rapidly identified which modulate the activity of Mlsn1.

This invention also relates to the use of the Mlsn1 gene product to screen for any molecule involved in the Mlsn1 gene pathway, i.e. for any protein or peptide that interact with Mlsn1. Such targets (or Mlsn1 partners or signaling molecules) can be screened by protein-protein interaction analysis techniques (double-hybrid system for instance), or any other method known to the skilled artisan.

Ligand Binding Screening Method (Phage Display, Flashplate)

Ligand binding screening comprises, preferably, contacting a test compound with an Mlsn1 gene product or Mlsn1 transcription promoter element(s) and determining the ability of said test compound to interact with the Mlsn1 gene product or transcription promoter element(s). In a typical embodiment, the ligand binding screen comprises contacting one or several (in parallel) test compounds with a Mlsn1 polypeptide (e.g., protein or a fragment thereof) or transcription promoter element(s), in the presence (or absence, as a reference) of a Mlsn1 ligand or transcription promoter ligand, and assessing the ability of the test compound(s) to bind Mlsn1 transcription polypeptide or promoter element(s) by assessing ligand binding.

The Mlsn1 polypeptide can be part of an intact cell, membrane preparation or a purified polypeptide, optionally attached to a support, such as beads, column plate, etc. The polypeptide is preferably characterized by comprising an amino acid sequence selected from SEQ ID NO1 to 22.

The Mlsn1 transcription promoter element(s) can preferably but not exclusively be a purified nucleic acid, optionally attatched to a support such as beads, column, plate etc. The nucleic acid encoding the promoter elements is preferably characterized by comprising nucleic acid sequences selected from SEQ ID NO41, 42 or 43.

The test compound can be a peptide or a protein or an antibody or chemical entity, either alone or in combination(s) or in a mixture with any other substance. The test compound may even represent a library of compounds. Optionally, excess non Mlsn1 bound ligand can be removed by separation. Separation can take the form of washing/filtering or centrifugation (to pellet the Mlsn1 protein). In this latter case, the supernatant can then be removed and the Mlsn1 re-suspended in buffer.

The binding is preferably performed in the presence of a Mlsn1 ligand or transcription promoter element ligand, to allow an assessment of the binding activity of each test compound. In particular, in a preferred embodiment, a labeled Mlsn1 ligand or promoter element ligand is used and the binding activity of the test compound is determined by assessing any modulation of the ligand binding.

The ligand may be contacted with the Mlsn I polypeptide or transcription promoter element either before, simultaneously or after the test compound.

The ligand should be detectable and/or quantifiable. To achieve this, the ligand can be labeled in a number of ways, such as with a chromophore, radioactive, fluorescent, phosphorescent, enzymatic or antibody label, for instance. If the ligand is not directly detectable it must be amenable to detection and quantification by secondary detection, which may employ the above technologies. Preferably (in the case of certain detection methods), unbound ligand is removed from the mixture prior to detecting ligand binding, as described above.

Alternatively the Mlsn1 polypeptide or fragment thereof of Mlsn1 transcription promoter element(s) can be detectable or quantifiable. This can be achieved in a similar manner to that described above.

Preferred examples of such Mlsn1 ligands that can be used in the instant invention include any product that is known to interact with Mlsn1, such as an antibody, physiological ligand or receptor, or synthetic ligands or Mlsn1 inhibitors. Specific examples of such ligands include pyrazole compounds as described in WO99/19303, which exhibit calcium release-dependent calcium channel inhibitory effect. These compounds may be labeled according to various techniques, including radioactivity or fluorescence. More specific compounds are compounds SEW04225, KM02940, KM03000 and GK02421 as listed in the Maybridge Co. Trial Drug Catalogue (UK, Cornwall, published August 95).

Preferred examples of Mlsn1 transcription promoter element ligands that can be used in this instant invention include any product that is known to interact with Misnl promoter elements, such as polymerases, antibodies, other naturally occuring ligands or synthetic ligands.

Binding of the test compound modifies the interaction of the ligand with the binding site and changes the affinity or binding of ligand for/to its binding site. The difference between the observed amount of ligand bound relative to the theoretical maximum amount of ligand bound (or to the ligand bound in the absence of a test compound under the same conditions) is a reflection of the binding ability (and optionally the amount and/or affinity) of a test compound to bind Mlsn1 or its transcription promoter elements.

Alternatively, the amount of test compound bound to the Mlsn1 protein or a fragment thereof, or transcription promoter element(s) can be determined by a combination of chromatography and mass spectroscopy. The amount of test compound bound to the Mlsn1 protein or a fragment thereof or transcription promoter element(s) can also be determined by direct measurement of the change in mass upon compound or ligand binding to Mlsn1 or transcription promoter element(s). This can be achieved with technologies such as Biacore (Amersham Pharmacia). Alternatively, the Mlsn1 protein or a fragment thereof, or promoter element(s), the compound or the ligand can be fluorescently labeled and association of Mlsn1 protein or promoter element(s) with the compound can be followed by changes in Fluorescence Energy Transfer (FRET).

Functional Screening Method

In addition to, or in replacement of the binding screen, functional screens may be used to identify or characterize immuno-modulators or Mlsn1 modulators. Such a functional screen is advantageously transferable to high throughput, to allow the screening of large numbers of test compounds. The functional screen essentially comprises contacting a Mlsn1-expressing cell with a test compound, and assessing the ability of said test compound to affect Mlsn1 gene pathway or Mlsn1 gene product activity within the cells. This can be performed by measuring calcium fluxes, activation potential (or profile) or signal transduction pathway (e.g., selected cellular gene expression level) within said cells.

In a particular embodiment, the present invention thus resides in a method of screening (or identifying or selecting or characterizing) immuno-modulators, comprising (i) exposing a Mlsn1-expressing cell to one or several test compounds and (ii) detecting the compound that induce a modulation of the calcium flux or the activation potential in said cells. More preferably, the cells are recombinant cells expressing a Mlsn1 polypeptide, or immune cells, in particular antigen-presenting cells, more preferably macrophages.

In a first preferred variant of this embodiment, the functional screening method comprises the following steps:

(1) obtaining a recombinant cell expressing Mlsn1 gene or gene product,

(2) exposing said recombinant cell to a substance or molecule to be tested; and

(3) measuring the change in calcium flux or activation potential within the exposed recombinant cell.

In a preferred embodiment, the cell express a functional Mlsn1 polypeptide comprising all or part of SEQ ID NO1, in particular a polypeptide comprising a sequence selected from SEQ ID NO1 to 22.

Calcium Flux

Calcium Flux may be measured according to various techniques, such as for instance using sodium sensitive dyes such as Fura II. In a particular embodiment, compounds are selected for their ability to cause an increase in calcium concentration within the cell. In an other embodiment, compounds are selected for their ability to cause a decrease in calcium concentration within the cell. Alternatively, compounds are selected for their ability to alter the rate of recovery of the calcium channel.

Electrophysiology

In another embodiment of the functional screening method, substances or molecules of interest are selected for their ability to induce changes in the activation potential, the inactivation time, or the rate of recovery of the sodium channel. Preferred molecules or substances are those inducing a decrease in the inactivation potential, and/or a decrease in the rate of inactivation, and/or which decreases the rate of recovery from inactivation, as compared with the same measures performed in the absence of the substance of molecule to be tested.

In an other preferred variant, the functional screen is based on a measure of selected gene expression or activity within the cells. In this regard, the present invention now proposes, for the first time, to select Icrac modulators based on their ability to modify the expression pattern of particular genes. More preferably, the selected genes are selected from cytokine genes (in particular interleukin-2, interleukin-8, etc.) and chemokine genes (in particular MIPa). Accordingly, a preferred screening method of this invention comprises contacting Mlsn1-expressing cells with one or several test compounds and identifying the compounds that cause a modification in expression or activity of a gene selected from a cytokine or a chemokine gene, within said exposed cells.

Modification in gene expression or activity may be determined by assessing the presence or (relative) level of the gene product in the cell, in particular of the mRNA or polypeptide. Polypeptides may be detected or dosed using affinity reagents, such as antibodies (or fragments or derivatives thereof), either within the cells or at the cell surface, or in the cell culture medium (or supernatant). mRNA levels may be evaluated using conventional techniques (primers, probes, etc). In a specific embodiment, the exposed cells (or any preparation derived therefrom such as membranes, pellets, RNAs, etc) are contacted with a support to which particular affinity reagents are attached (antibodies or nucleic acids).

Transcription promoter-reporter

In a further embodiment, a promoter-reporter assay can be used to screen for compounds that modulate Mlsn1 expression. A reporter gene encoding a reporter molecule (i.e. a molecule(s) that can be directly or indirectly detected) is placed 3′ with respect to the nucleic acids encoding Mlsn1 transcription promoter element(s). The position and orientation of which is such that reporter gene expression is initiated from the Mlsn1 transcription promoter. Preferable transcription promoter elements are chosen from nucleic acids encoded by SEQ ID NO41, 42 or 43. Preferred reporter genes could be those that encode functional β-Galactosidase, Green fluorescence Protein or Luciferase. However, many other reporter molecules could be used, the identity and characteristics of which are known to those skilled in the art.

Further optimization of reporter expression could be achieved with the inclusion of additional nucleic acid elements, as described previously.

The action of test compounds can be measured by exposure of these compounds to cell lines expressing the promoter-reporter construct. Compounds which down regulate the action of the Mlsn1 promoter elements will manifest by reducing the amount or activity of the reporter in the cell relative to cells not compound treated cells. Compounds which up regulate the action of the Mlsn1 promoter elements will manifest by increasing the amount or activity of the reporter in the cell relative to cells not compound treated cells.

Compounds which modulate the amount of reporter or activity by other means could be distinguished from those that do not by secondary screening with similar cell lines expressing reporter with non Mlsn1 transcription promoter elements. Compounds which modulate the reporter in the first screen and not in the second screen could be considered to mediate its action either directly or indirectly through the Mlsn1 transcription promoter elements.

An assay would comprise the following steps;

a) obtaining a recombinant cell expressing a reporter molecule under the control of an Mlsn1 transcription promoter Mlsn1 gene,

b) exposing said recombinant cell to a substance or molecule to be tested; and

Measuring the change in reporter expression or activity in said recombinant cell.

According to a further embodiment, the active compounds are evalued by their in vitro activity. This activity is determined for example by IC 50 measurement.

The choice of compounds (called in-after hit) from which series of compounds are developped should take into account parameters such as IC 50 measurement, structural features of the hits, the possibility to alter the substituants, the physico-chemical properties such as solubility as well as physiological features such as half-life and bioavailability . . . Therefore another embodiment of the instant invention is the use of an active compound (hit) as identified, selected or characterized by the screening method described previously for the preparation of pharmaceutical compounds or compositions that bind and/or modulate the biological activity of Mlsn1.

Diagnostics Assays and Kits

The inventors believe that the ability to detect and measure the amount of Mlsn1 in immune cells would be a valuable tool for the prognosis of aberrant Mlsn1 and Icrac conditions in humans. The inventors believe that detection of Mlsn1 in immune cells could be achieved by measuring the amount of expressed Mlsn1 nucleic acid in immune cells. This could be achieved using a multi-step process:

(a) obtaining a (small) blood sample from a patient (of known volume),

(b) optionally separating immune cells from other blood components. This could be achieved by centrifugation and/or affinity chromatography.

(c) reverse transcribe cellular RNA to cDNA using protocols similar to those described in examples,

(d) amplify Mlsn1 nucleic acid obtained in step c) by PCR with a plurality of Mlsn1 specific oligonucleotides capable of hybridizing, under stringent conditions to Mlsn1 nucleic acids. Such oligonucleotides and amplification conditions have been outlined in examples,

(e) characterize the PCR product. One way to identify the presence of an Mlsn1 specific PCR product would be to electrophorese an aliquote of amplified nucleic acid in agarose. Such technology has been outlined in examples.

This test could be used to determine if Mlsn1 is being expressed in immune cells, therefore providing a valuable indication in the prognosis of disorders associated with aberrant Icrac function. Furthemore, an ability to accurately quantitate the level of expression if Mlsn1 in immune cells would help in the prognosis of the severity of an Icrac associated disorder. Quantitation could be achieved with the inclusion of further step(s). This step(s) would require the assay of a known quantity of nucleic acid which encodes Mlsn1.

Co-electrophoresis of samples which have not been subjected to blood products but have been “spiked” with known concentrations of nucleic acids encoding human Mlsn1 such as SEQ ID NO23 or fragments thereof, or complementary sequences thereto with the test sample, would allow identification and quantitation of levels of Mlsn1 in the blood sample.

Other aspects and advantages of the present invention will be disclosed in the following experimental section, which should be regarded as illustrative and not limiting the scope of protection.

### EXAMPLES

1. A Mlsn1 Gene Product is Expressed in Human U937 Cells

cDNA libraries were prepared from U937 cells (these cells being refered to as undifferentiated cells) and dbcAMP differentiated U937 cells (48 hours) using the Clontech SMART system (K1052-1). In brief, U937 cell were cultured in suspension in cell culture media (RPMI 1640 plus 10% heat inactivated FBS) at 37° C. in 5% CO₂, in the presence and absence of differentiating agent (dbcAMP Conc 1 μM) (48 hours). Differentiated and undifferentiated cells were harvested by centrifugation at 1,200 g for 10 minutes, the supernatent removed from the cell pellet and cellular RNA extracted using FastTrack 2.0 kit (Invitrogen). A SMART library was prepared from the differentiated and undifferentiated cells according to Clontech protocols. These libraries were used to determine the relative abundance of Mlsn1 cDNA in the libraries.

Two nested primer pairs were designed to screen for Mlsn1 cDNA in the above libraries. The sequence of these primers is as follows:

| | | |
|---|---|---|
| Primer 1a | (SEQ ID N^{o} 44): | TGAAGTAAAATCAATATCCAACCAGG |
| Primer 1b | (SEQ ID N^{o} 45): | GCACTGCTCCTCGAACTCATGCAGCC |
| Primer 2a | (SEQ ID N^{o} 46): | TCCAACCAGGTGTGGAAGTTCCAGCG |
| Primer 2b | (SEQ ID N^{o} 47): | GGAAGTGCTCCTGCACGCACTGCTCC |

Under the following PCR conditions:

All PCR reactions were carried out using Advantage cDNA PCR Kit (#K1905-1) in the suppliers buffer conditions. PCR primer concentration was 20 fmol/primer/100 μl reaction. cDNA template concentration was 0.2 μg. PCR reactions were carried out as two step cycles (denaturation 95° C. for 30 sec, for annealing/elongation 68° C. for 6 min). PCR product was visualised by electrophoresis in an agarose gel and staining with ethidium bromide (0.5 μg/ml).

Nested PCR using primer pair SEQ ID NO44 and 47 and nested pair SEQ ID NO45 and 46 indicated Mlsn1 expression in U937 cells. Similar experiments indicated the expression of Mlsn1 in Jurkat and KU-812 cells.

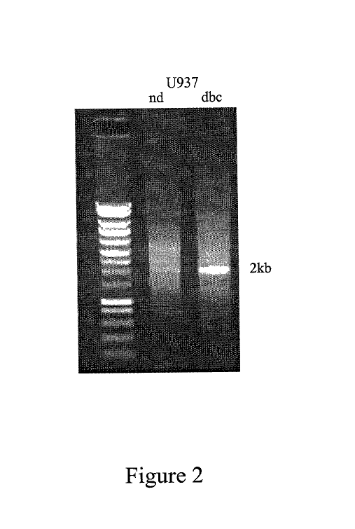

The presence of the expression of Mlsn1 in these libraries was further confirmed using nested PCR primers, SEQ ID NO: 44 and 46 together with CDS III/3 primer of SMART cDNA libraries (c.f. Advantage cDNA PCR Kit (#K1905-1)). A 2 kb PCR product was obtained (FIG. 2). This corresponded to the predicted size of a Mlsn1 PCR product and indicated that the gene encoding Mlsn1 was being expressed in differentiated and undifferentiated U937 cell lines. However, the intensity of the band corresponding to the Mlsn1 PCR product was greater in the differentiated cell library than in the undifferentiated cell library. These results indicate that Mlsn1 cDNA was present in U937 cell lines and that this Mlsn1 expression was more pronounced in U937 cell lines differentiated to a macrophage phenotype.

2. MLSN1 (TrpC8) Expression is Differentially Increased in dbcAMP Treated (Macrophage-like) U937 Cells

The relative abundance of nucleic acid encoding Mlsn1 in undifferentiated and dbcAMP differentiated U937 cell lines was determined. Briefly, undifferentiated and dbcAMP differentiated U937 cell lines were cultured and harvested as described above, and subtracted cDNA libraries prepared using Clontech PCR Select kit (K1804-1) and the protocols therein. This technology allows for selective enrichment of differentially expressed genes between the differentiated and undifferentiated cell lines.

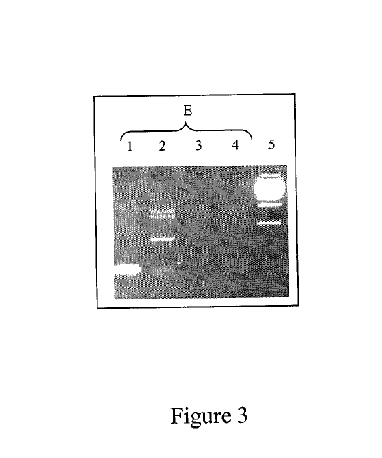

Following PCR substraction, Mlsn1 specific primer pair (SEQ ID NO44 and 47) were used to amplify cDNA-fragments from Mlsn1 from subtracted pools and controls using Advantage cDNA PCR, under suppliers conditions as two step cycles (95° C. for 30 sec, 68° C. for 1 min annealing/elongation) (FIG. 3).

Forward subtraction of differentiated cells nucleic acid with non differentiated cell nucleic acid resulted in a strong Mlsn1 specific PCR product. Back subtraction of non-differentiated cells with differentiated cells resulted in undetectable amounts PCR product. This result indicates that there was greater Mlsn1 gene expression in the differentiated U937 cells than in the undifferentiated U937 cells. This result correlates with the observation that the Icrac phenotype increases during dbcAMP treatment of U937 cell line to a more macrophage like phenotype. The inventors conclude that the emergence of an Icrac phenotype in the dbcAMP differentiated U937 cells corresponds with an increase in expression of Mlsn1.

3. Down Regulation of Calcium Entry in dbcAMP Treated (Macrophage-like) U937 Cells Using Mlsn1 Anti-sense

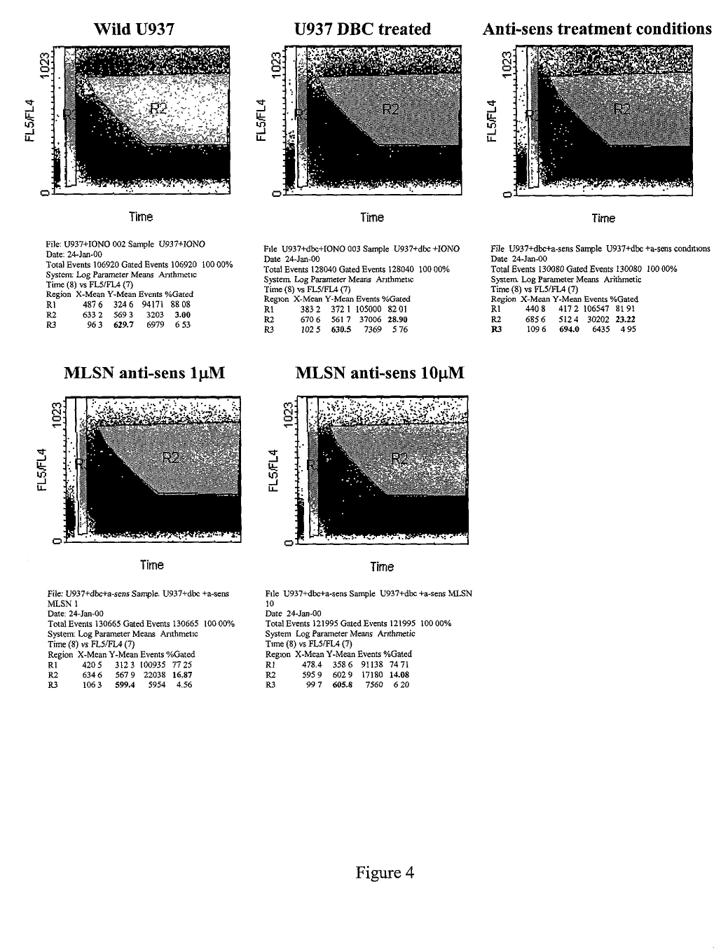

U937 cells were cultured at 37° C. in 5% CO₂ in RPMI 1640, 10% heat inactivated FBS. Cells were incubated in absence or presence of 0, 1 or 10 μM Mlsn1 anti-sense nucleic acid (SEQ ID NO:26), 4 hours prior to and 48 h during differentiation treatment by dbcAMP. All oligonucleotides used in this experiment contained phosphorothiorate modifications at their first two and last two linkages (terminal 5′ and 3′ linkage). A scrambled oligonucleotide (⁵′CTGGTGGAAGAAGAGGACGTCCAT^{3′}, SEQ ID NO48) was used at 10 μM in the same conditions as negative control. Another oligonucleotide against FcγRII (CD 32) (⁵′TCTGGGACATACATTCTGAGACAT^{3′}, SEQ ID NO49) was used at 10 μM in the same conditions as positive control for anti-sense uptake. DbcAMP induced differentiation was controlled by immunofluorescence using FACS quantitation of CD 32 and CD 64, as well as Ca²⁺ signaling after Thapsigargin stimulation, before and after dbcAMP treatment using fura 2 and fluorimetry. Intra-cellular Ca²⁺ after stimulation by ionomycin was measured during FACS analysis. Cells were loaded with 2 μg/ml indo-1 during 30 min at 37° C., 5% CO₂ in RPMI, 2 mg/ml BSA. Cells were then activated by 2 μg/ml ionomycin. Ionomycin induced variations of intra-cellular calcium concentration were measured by flow cytometry (FIG. 4). CD 32 expression was quantitated by immunofluoresence with an FITC labeled anti CD32 antibody using flow cytometry.

Comparison of FIGS. 4a) and 4 b) shows apparition of a long lasting calcium current when differentiating U937 cells by dbcAMP.

Comparison of FIGS. 4b) and 4c) shows a non specific inhibition of long lasting calcium current due to antisens treatment conditions.

Comparison of FIGS. 4c) and 4d) show a specific inhibition of long lasting calcium current due to 1 μM Mlsn1 antisens.

Comparison of FIGS. 4c) and 4e) show a specific inhibition of long lasting calcium current due to 10 μM Mlsn1 antisens.

Dibutyrate cAMP differentiation induced differences in calcium flux responses on U937 cells after Ionomycine 2 μg/ml stimulation (R2 were 3% in wild type Vs 29% after DBC differentiation. Particularly, Even though anti-sens condition (absence of anti-sens itself) affected the level of differentiation (R2 were 29% Vs 23%), Mlsn1 anti-sens treatment seems to decrease the expression of R2 population in a dose-related manner, R2 moved from 23% to 17% and 14% according to anti-sens control, 1 μM and 10 μM Mlsn1 anti-sens treatment respectively.

These data show the existence of a long lasting calcium flux after DBC differentiation of U937 cells with 39% inhibition of this typical calcium flux induction after Mlsn1 anti-sens 10 μtreatment.

DbcAMP treatment was shown to increase intra-cellular Ca²⁺ after stimulation according to Floto et al. The results demonstrate that anti-sense oligonucleotide treated cultures have their calcium signal decreased by about 40%. This antisense experiment demonstrates that the down-regulation of Mlsn1 expression anti-sense oligonucleotides results in a diminished Icrac phenotype in U937 cell lines. This result also demonstrates that anti-sense nucleic acid specific to Mlsn1 is capable of down-regulating the Icrac phenotype.

### REFERENCE LIST

1. Lewis, R. S. 1999. Store-operated calcium channels. Adv Second Messenger Phosphoprotein Res 33:279.

2. Berridge, M. J. 1993. Inositol trisphosphate and calcium signalling. Nature 361:315.

3. Streb, H., R. F. Irvine, M. J. Berridge, and I. Schulz. 1983. Release of Ca2+ from a nonmitochondrial intra-cellular store in pancreatic acinar cells by inositol-1,4,5-trisphosphate. Nature 306:67.

4. Putney, J. W. J. 1990. Capacitative calcium entry revisited. Cell Calcium 11:611.

5. Penner, R., G. Matthews, and E. Neher. 1988. Regulation of calcium influx by second messengers in rat mast cells. Nature 334:499.

6. Lewis, R. S. and M. D. Cahalan. 1989. Mitogen-induced oscillations of cytosolic Ca2+ and transmembrane Ca2+ current in human leukemic T-cells. Cell Regul 1:99.

7. Thastrup, O., P. J. Cullen, B. K. Drobak, M. R. Hanley, and A. P. Dawson. 1990. Thapsigargin, a tumor promoter, discharges intra-cellular Ca2+ stores by specific inhibition of the endoplasmic reticulum Ca²⁺-ATPase. Proc Natl Acad Sci USA 87:2466.

8. Gouy, H., D. Cefai, S. B. Christensen, P. Debre, and G. Bismuth. 1990. Ca2+ influx in human T lymphocytes is induced independently of inositol phosphate production by mobilization of intra-cellular Ca²⁺ stores. A study with the Ca²⁺ endoplasmic reticulum-ATPase inhibitor thapsigargin. Eur J Immunol 20:2269.

9. Mason, M. J., M. P. Mahaut-Smith, and S. Grinstein. 1991. The role of intra-cellular Ca2+ in the regulation of the plasma membrane Ca²⁺ permeability of unstimulated rat lymphocytes. J Biol Chem 266:10872.

10. Hoth, M. and R. Penner. 1992. Depletion of intra-cellular calcium stores activates a calcium current in mast [see comments]. Nature 355:353.

11. Hoth, M. and R. Penner. 1993. Calcium release-activated calcium current in rat mastcells. J Physiol (Lond) 465:359.

12. Zweifach, A. and R. S. Lewis. 1993. Mitogen-regulated Ca²⁺ current of T lymphocytes is activated by depletion of intra-cellular Ca²⁺ stores. Proc Natl Acad Sci USA 90:6295.

13. Parekh, A. B. and R. Penner. 1997. Store depletion and calcium influx. Physiol Rev 77:901.

14. Clapham, D. E. 1995. Calcium signaling. Cell 80:259.

15. Dolmetsch, R. E., K. Xu, and R. S. Lewis. 1998. Calcium oscillations increase the efficiency and specificity of gene expression [see comments]. Nature 392:933.

16. Li, W., J. Llopis, M. Whitney, G. Zlokarnik, and R. Y. Tsien. 1998. Cell-permeant caged InsP3 ester shows that Ca2+ spike frequency can optimize gene expression [see comments]. Nature 392:936.

17. Partiseti, M., F. Le Deist, C. Hivroz, A. Fischer, H. Korn, and D. Choquet. 1994. The calcium current activated by T-cell receptor and store depletion in human lymphocytes is absent in a primary immunodeficiency. J Biol Chem 269:32327.

18. Floto R A, Mahaut-Smith M P, Allen J M, Somasundaram B. 1996. Differentiation of the human monocytic cell line U937 results in an upregulation of the calcium release-activated current, ICRAC. J Physiol. (Lond). 495:331.

19. Melendez A, Floto R A, Cameron A J, Gillooly D J, Harnett M M, Allen J M. 1998. A molecular switch changes the signalling pathway used by the Fc gamma RI antibody receptor to mobilise calcium. Curr Biol. 4:210.

1. A method for the screening of Mlsn1 modulators which comprises the use of any purified or isolated nucleic acid encoding a human Mlsn1 or a sequence complementary thereto, wherein the encoding nucleic acid encodes a polypeptide having the following characteristics:
(1) the N-terminal region is encoded by a consecutive sequence of at least 75 nucleotides, located 5′ with respect to nucleotides encoding the transmembrane and C-terminal regions of Mlsn1 protein,
(2) a transmembrane region encoding six membrane spanning domains, encoded by a consecutive sequence of at least 150 nucleotides, located 3′ with respect to the nucleotides encoding the N-terminal region and 5′ with respect to nucleotides encoding the C-terminal region, and
(3) a C-terminal region encoded by a consecutive sequence of at least 75 nucleotides, located 3′ with respect to nucleotides encoding the N-terminal region and transmembrane region.

2. The method of claim 1, wherein said nucleic acid has at least 80% nucleotide identity with the nucleotide sequence of SEQ ID NO23 or a sequence complementary thereto.

3. A method for the screening of Mlsn1 modulators which comprises the use of any purified or isolated human Mlsn1 polypeptide or a fragment thereof, wherein the polypeptide has the following characteristics:
(1) the N-terminal region comprises a consecutive sequence of at least 25 amino acids, located N-terminal with respect to the transmembrane and C-terminal regions of Mlsn1 protein,
(2) a transmembrane region forming six membrane spanning domains, comprising a consecutive sequence of at least 50 amino acids, located C-terminal with respect to the N-terminal region and N-terminal with respect to the C-terminal region, and
(3) a C-terminal region comprising a consecutive sequence of at least 25 amino acids, located C-terminal with respect to the N-terminal and transmembrane regions.

4. The method of claim 3, wherein said polypeptide has at least 90% amino identity with the sequence of SEQ ID NO1 or a fragment thereof.

5. A method for the diagnosis of disorders associated with aberrant Icrac function in immune cells which comprises the use of any purified or isolated nucleic acid encoding a human Mlsn1 or a sequence complementary thereto for, wherein the encoding nucleic acid encodes a polypeptide having the following characteristics:
(1) the N-terminal region is encoded by a consecutive sequence of at least 75 nucleotides, located 5′ with respect to nucleotides encoding the transmembrane and C-terminal regions of Mlsn1 protein,
(2) a transmembrane region encoding six membrane spanning domains, encoded by a consecutive sequence of at least 150 nucleotides, located 3′ with respect to the nucleotides encoding the N-terminal region and 5′ with respect to nucleotides encoding the C-terminal region, and
(3) a C-terminal region encoded by a consecutive sequence of at least 75 nucleotides, located 3′ with respect to nucleotides encoding the N-terminal region and transmembrane region.

6. The method of claim 5, wherein said nucleic acid has at least 80% nucleotide identity with the nucleotide sequence of SEQ ID NO23 or a sequence complementary thereto.

7. A method for the diagnosis of disorders associated with aberrant Icrac function in immune cells which comprises The use of any purified or isolated polypeptide encoding a human Mlsn1 or fragment thereof, wherein the polypeptide has the following characteristics:
(1) the N-terminal region comprises a consecutive sequence of at least 25 amino acids, located N-terminal with respect to the transmembrane and C-terminal regions of Mlsn1 protein,
(2) a transmembrane region forming six membrane spanning domains, comprising a consecutive sequence of at least 50 amino acids, located C-terminal with respect to the N-terminal region and N-terminal with respect to the C-terminal region, and
(3) a C-terminal region comprising a consecutive sequence of at least 25 amino acids, located C-terminal with respect to the N-terminal and transmembrane regions.

8. The method of claim 7, wherein said polypeptide has at least 90% amino identity with the sequence of SEQ ID NO1 or a fragment thereof.

9. A purified or isolated human Mlsn1 N-terminal polypeptide region, wherein the N-terminal region comprises a consecutive sequence of at least 25 amino acids, located N-terminal of the first transmembrane region of the Mlsn1 protein.

10. A purified or isolated human Mlsn1 N-terminal polypeptide region according to claim 9, wherein said N-terminal polypeptide has at least 90% amino acid identity with any of the amino acid sequences SEQ ID NO2 or 3, or fragments thereof.

11. A purified or isolated human Mlsn1 C-terminal polypeptide region, wherein the C-terminal region comprises a consecutive sequence of at least 25 amino acids, located C-terminal of the sixth transmembrane region of the Mlsn1 protein.

12. A purified or isolated human Mlsn1 C-terminal region according to claim 11, wherein said C-terminal polypeptide has at least 90% amino acid identity with any of the polypeptides of amino acid sequences SEQ ID NO4 or 5, or fragments thereof.

13. A purified or isolated human Mlsn1 Intra-cellular loop-1 polypeptide region, wherein the intra-cellular loop-1 region comprises a consecutive sequence of at least 7 amino acids, located C-terminal of the second transmembrane region and N-terminal to the third transmembrane region of the Mlsn1 protein.

14. A purified or isolated human Mlsn1 intracellular loop-1 region according to claim 13, wherein said intra-cellular loop-1 polypeptide has at least 90% amino acid identity with any of the polypeptides of amino acid sequences SEQ ID NO6 or 7, or fragments thereof.

15. A purified or isolated human Mlsn1 intra-cellular loop-2 polypeptide region, wherein the intra-cellular loop-2 polypeptide region comprises a consecutive sequence of at least 7 amino acids, located C-terminal of the fourth transmembrane region and N-terminal of the fifth transmembrane region of the Mlsn1 protein.

16. A purified or isolated human Mlsn1 intra-cellular loop-2 region according to claim 15, wherein said intra-cellular loop-2 region polypeptide has at least 90% amino acid identity with any of the polypeptides of amino acid sequences SEQ ID NO8 or 9, or fragments thereof.

17. A purified or isolated human Mlsn1 extra-cellular loop-1 polypeptide region, wherein the extra-cellular loop-1 region comprises a consecutive sequence of at least 5 amino acids, located C-terminal of the first transmembrane region and N-terminal of the second transmembrane region of the Mlsn1 protein.

18. A purified or isolated human Mlsn1 extra-cellular loop-1 region according to claim 17, wherein said extra-cellular loop-1 region polypeptide has at least 90% amino acid identity with any of the polypeptides of amino acid sequences SEQ ID NO10 or 11, or fragments thereof.

19. A purified or isolated human Mlsn1 extra-cellular loop-2 polypeptide region, wherein the extra-cellular loop-2 region comprises a consecutive sequence of at least 5 amino acids, located C-terminal of the third transmembrane region and N-terminal of the fourth transmembrane region of the Mlsn1 protein.

20. A purified or isolated human Mlsn1 extra-cellular loop-2 region according to claim 19, wherein said extra-cellular loop-2 region polypeptide has at least 90% amino acid identity with any of the polypeptides of amino acid sequences SEQ ID NO12 or 13, or fragments thereof.

21. A purified or isolated human Mlsn1 extra-cellular loop-3 polypeptide region, wherein the extra-cellular loop-3 region comprises a consecutive sequence of at least 7 amino acids C-terminal of the fifth transmembrane region and N-terminal of the sixth transmembrane region of the Mlsn1 protein.

22. A purified or isolated human Mlsn1 extra-cellular loop-3 region according to claim 21, wherein said extra-cellular loop-3 region polypeptide has at least 90% amino acid identity with any of the polypeptides of amino acid sequences SEQ ID NO14, 15, 16 or 17, or fragments thereof.

23. A purified or isolated human Mlsn1 polypeptide containing both intra- and extra-cellular loop regions, wherein said intra- and extra-cellular loop region comprises a consecutive sequence of at least 50 amino acids, located C-terminal to the N-terminal region and N-terminal to the C-terminal region of the Mlsn1 protein.

24. A purified or isolated human Mlsn1 peptide containing both intra- and extra-cellular loop regions according to claim 23, wherein said intra- and extra-cellular loop region polypeptide has at least 90% amino acid identity with any of the polypeptides of amino acid sequences SEQ ID NO18 or 19, or fragments thereof.

25. A purified or isolated nucleic acid encoding the human Mlsn1 N-terminal region, wherein the N-terminal region is encoded by a consecutive sequence of at least 75 nucleotides, located 5′ with respect to nucleotides encoding the first transmembrane region of the Mlsn1 protein.

26. A purified or isolated nucleic acid encoding the human Mlsn1 N-terminal region according to claim 25, wherein said nucleic acid has at least 80% nucleic acid identity with any of the nucleotide sequences SEQ ID NO24 or 25, or a complementary sequence thereto.

27. A purified or isolated nucleic acid encoding the human Mlsn1 C-terminal region, wherein the C-terminal region is encoded by a consecutive sequence of at least 75 nucleotides, located 5′ with respect to nucleotides encoding the sixth transmembrane region of the Mlsn1 protein.

28. A purified or isolated nucleic acid encoding the human Mlsn1 C-terminal region according to claim 27, wherein said nucleic acid has at least 80% nucleic acid identity with any of the nucleotide sequences of SEQ ID NO26 or 27, or a complementary sequence thereto.

29. A purified or isolated nucleic acid encoding the human Mlsn1 intra-cellular loop-1 region, wherein the intra-cellular loop-1 region is encoded by a consecutive sequence of at least 21 nucleotides, located 3′ with respect to nucleotides encoding the second transmembrane region and 5′ with respect to nucleotides encoding the third transmembrane region of the Mlsn1 protein.

30. A purified or isolated nucleic acid encoding the human Mlsn1 intra-cellular loop-1 region according to claim 29, wherein said nucleic acid has at least 80% nucleic acid identity with any of the nucleotide sequences of SEQ ID NO28 or 29, or a complementary sequence thereto.

31. A purified or isolated nucleic acid encoding the human Mlsn1 intra-cellular loop-2 region, wherein the intra-cellular loops-2 region is encoded by a consecutive sequence of at least 21 nucleotides, located 3′ with respect to nucleotides encoding the fourth transmembrane region and 5′ with respect to nucleotides encoding the fifth transmembrane region of the Mlsn1 protein.

32. A purified or isolated nucleic acid encoding the human Mlsn1 intra-cellular loop-2 region according to claim 31, wherein said nucleic acid has at least 80% nucleic acid identity with any of the nucleotide sequences of SEQ ID NO30 or 31, or a complementary sequence thereto.

33. A purified or isolated nucleic acid encoding the human Mlsn1 extra-cellular loop-1 region, wherein the extra-cellular loop-1 region is encoded by a consecutive sequence of at least 15 nucleotides, located 3′ with respect to nucleotides encoding the first transmembrane region and 5′ with respect to nucleotides encoding the second transmembrane region of the Mlsn1 protein.

34. A purified or isolated nucleic acid encoding the human Mlsn1 extra-cellular loop-1 region according to claim 33, wherein said nucleic acid has at least 80% nucleic acid identity with any of the nucleotide sequences of SEQ ID NO32 or 33, or a complementary sequence thereto.

35. A purified or isolated nucleic acid encoding the human Mlsn1 extra-cellular loop-2 region, wherein the extra-cellular loop-2 region is encoded by a consecutive sequence of at least 15 nucleotides, located 3′ with respect to nucleotides encoding the third transmembrane region and 5′ with respect to nucleotides encoding the fourth transmembrane region of the Mlsn1 protein.

36. A purified or isolated nucleic acid encoding the human Mlsn1 extra-cellular loop-2 region according to claim 35, wherein said nucleic acid has at least 80% nucleic acid identity with any of the nucleotide sequences of SEQ ID NO34 or 35, or a complementary sequence thereto.

37. A purified or isolated nucleic acid encoding the human Mlsn1 extra-cellular loop-3 region, wherein the extra-cellular loop-3 region is encoded by a consecutive sequence of at least 21 nucleotides, located 3′ with respect to nucleotides encoding the fifth transmembrane region and 5′ with respect to nucleotides encoding the sixth transmembrane region of the Mlsn1 protein.

38. A purified or isolated nucleic acid encoding the human Mlsn1 extra-cellular loop-3 region according to claim 37, wherein said nucleic acid has at least 80% nucleic acid identity with any of the nucleotide sequences of SEQ ID NO36, 37 or 38, or a complementary sequence thereto.

39. A purified or isolated nucleic acid encoding a human Mlsn1 polypeptide containing both intra- and extra-cellular loop regions, wherein said intra- and extra-cellular loop region is encoded by a consecutive sequence of at least 150 nucleotides, located 3′ with respect to nucleotides encoding the N-terminal region and 5′ with respect to nucleotides encoding the C-terminal regions of the Mlsn1 protein.

40. A purified or isolated nucleic acid encoding the human Mlsn1 polypeptide containing both intra- and extra-cellular loop regions according to claim 39, wherein said nucleic acid has at least 80% nucleic acid identity with any of the nucleotide sequences of SEQ ID NO39 or 40, or a complementary sequence thereto.

41. A nucleic acid sequence encoding an Mlsn1 transcription promoter of SEQ ID NO41 or a complementary sequence thereto.

42. A nucleic acid sequence of claim 41, having at least 80% nucleotide identity with the nucleotide sequence of SEQ ID NO41 or a complementary sequence thereto.

43. A nucleic acid sequence encoding an Mlsn1 transcription promoter of SEQ ID NO42 or a complementary sequence thereto.

44. A nucleic acid sequence of claim 43, having at least 80% nucleotide identity with the nucleotide sequence of SEQ ID NO42 or a complementary sequence thereto.

45. A nucleic acid sequence encoding an Mlsn1 transcription promoter of SEQ ID NO43 or a complementary sequence thereto.

46. A nucleic acid sequence of claim 45, having at least 80% nucleotide identity with the nucleotide sequence of SEQ ID NO43 or a complementary sequence thereto.

47. A recombinant vector comprising a nucleic acid according to any one of the claims 25 to 46.

48. A recombinant vector comprising a nucleic acid encoding an Mlsn1 polypeptide according to any one of the claims 9 to 24.

49. A recombinant host cell comprising a nucleic acid according to any one of the claims 25 to 46 or a vector of claim 47 or 48.

50. A recombinant host cell comprising a nucleic acid ncoding an Mlsn1 polypeptide according to any one of the claims 9 to 24.

51. A method for producing a Mlsn1 polypeptide, wherein the said method comprises the following steps of:
a) culturing, in an appropriate culture medium, a host cell previously transformed or transfected with a polynucleotide according to any one of claims 25 to 48,
b) harvesting the culture medium thus conditioned or lyse the host cell, for example by sonication or by osmotic shock; and
c) separating or purifying, from said culture medium, or from the pellet of the resulting cell lysate, the produced Mlsn1 polypeptide of interest.

52. A method for screening ligand substances or molecules that are able to bind to a Mlsn1, or fragment thereof, or Mlsn1 transcription promoter elements of any one of the claims 1 to 46, said method comprising;
a) contacting a test compound with Mlsn1 gene, an Mlsn1 gene product or Mlsn1 transcription promoter element and,
b) measuring the ability of said test compound to interact with the Mlsn1 gene, Mlsn1 gene product or transcription promoter element (i.e., binding assay).

53. A method for screening ligand substances or molecules that are able to modulate the biological activity of Mlsn1, or fragment thereof, of any one of the claims 1 to 40, said method comprising;
a) contacting a test compound with an Mlsn1 expressing cell, and,
b) determining the ability of said test compound to affect Mlsn1 gene or an Mlsn1 gene product activity within the cells.

54. A method for screening ligand substances or molecules that are able to modulate the biological activity of Mlsn1, or fragment thereof, of any one of the claims 1 to 40, said method comprising;
a) obtaining a recombinant cell expressing Mlsn1 gene or gene product,
b) exposing said recombinant cell to a substance or molecule to be tested; and
c) Measuring the change in calcium flux or activation potential within the exposed recombinant cell.

55. A method for screening ligand substances or molecules that are able to modulate the biological activity of Mlsn1, or fragment thereof, of any one of the claims 41 to 46, said method comprising;
a) obtaining a recombinant cell expressing a reporter molecule under the control of an Mlsn1 transcription promoter Mlsn1 gene,
b) exposing said recombinant cell to a substance or molecule to be tested; and
c) measuring the change in reporter expression or activity in said recombinant cell.

56. A method for screening ligand substances or molecules that are able to bind to a Mlsn1, or modulate the biological activity of Mlsn1, according to any one of claims 51 to 55, wherein a competitor Mlsn1 binding ligand, preferably selected from the class of pyrazol compounds such as SEW04225, KM02940, KM03000 or GK02421, is added and the binding of said competitor in the presence and/or absence of a test compound is determined.

57. A method for detecting any one of the nucleic acids of claims 6, 8, or 25 to 50, in a blood sample, said method comprising;
a) obtaining a blood sample,
b) optionally separating immune cells from other blood components,
c) reverse transcribing cellular RNA to cDNA, and
d) amplifying Mlsn1 nucleic acid obtained in step c) by PCR with a plurality of Mlsn1 specific oligonucleotides capable of hybridizing, under stringent conditions to Mlsn1 nucleic acids and characterize the PCR product.

58. A method to screen compounds that modulate the activity of an antigen presenting cell, particularly of macrophages, in particular macrophage maturation which comprises the use of a Mlsn1 gene, gene product or transcriptional element.

59. The use of a compound that modulates the activity of Mlsn1 gene, gene product or transcriptional element, for the manufacture of a composition to regulate the activity of an antigen presenting cell, particularly of macrophages, in particular macrophage maturation.

60. An antisense nucleic acid molecule comprising a sequence region which is complementary to at least a region of a Mlsn1 nucleic acid of claims 25 to 40.

61. The use of an active compound as identified, selected or characterized by the screening method described in claims 52 to 56 for the preparation of pharmaceutical compounds or compositions that bind and/or modulate the biological activity of Mlsn1.
